(19) United States
(12) Patent Application Publication
(10) Pub. No.: US 2025/0361505 A1
(43) Pub. Date: Nov. 27, 2025

(54) COMPOSITIONS FOR AND METHODS OF GENE EDITING

(71) Applicants: President and Fellows of Harvard College, Cambridge, MA (US); Joslin Diabetes Center, Inc., Boston, MA (US)

(72) Inventors: Amy J. Wagers, Cambridge, MA (US); Alborz Karimzadeh, Boston, MA (US); Michael Florea, Cambridge, MA (US)

(21) Appl. No.: 18/867,708

(22) PCT Filed: May 22, 2023
(86) PCT No.: PCT/US2023/023160

§ 371 Date: Nov. 20, 2024

Related U.S. Application Data
(60) Provisional application No. 63/344,328, filed on May 20, 2022.
(60) Provisional application No. 63/460,170, filed on Apr. 18, 2023.

(51) Int. Cl. C12N 15/113; C12N 9/22; C12N 15/86; C12N 15/88

(52) U.S. Cl. CPC C12N 15/113; C12N 9/226; C12N 15/86; C12N 15/88; C12N 2310/20; C12N 2320/31; C12N 2750/14143

(57) ABSTRACT

Compositions and methods for treating a blood disorder in a subject comprising delivering a nucleic acid molecule including a nucleotide sequence encoding two to six guide RNAs (gRNAs) into a hematopoietic stem cell (HSC), a hematopoietic progenitor cell (HPC), or a population of hematopoietic stem and progenitor cells (HSPCs) are described.

### RELATED APPLICATIONS

This application claims the benefit of and priority to U.S. Provisional patent application Ser. No. 63/344,328, filed on May 20, 2022, and to U.S. Provisional patent application Ser. No. 63/460,170, filed on Apr. 18, 2023. The entire teachings of the above applications are incorporated herein by reference.

### BACKGROUND OF THE INVENTION

Sequence-targeting nuclease such as CRISPR/Cas9 provide powerful tools to edit mammalian genomes by engaging cellular mechanisms of DNA double strand break (DSB) repair. Hematopoietic stem and progenitor cells (HSPCs) are the self-renewing, multipotent, and engraftable source of all cells in the blood system. Genetic defects in the hematopoietic system can result in an array of diseases, including blood cancers, immune deficiency, autoimmunity, and hemoglobinopathy. As HSPCs have the potential to reconstitute the entire blood system, therapeutically gene-corrected HSPCs hold the promise of combating any genetic hematological disorder. However, efforts have focused on ex vivo gene editing of extracted HSPCs followed by transplantation of modified HSPCs back into the host. Various complications with ex vivo gene editing of HSPCs make this approach a last-resort option in many cases. For example, removal of HSPCs carries significant risks of graft failure and infection and require expensive procedures for handling the ex vivo cells. Further, ex vivo culturing of HSPCs is associated with the loss of “stemness” of HSPCs. Thus, there remains a need in the field for efficient in vivo gene editing of HSPCS.

### SUMMARY OF THE INVENTION

As described herein, adeno-associated virus (AAV) were used to deliver CRISPR guides to gene edit HSCs in vivo in a reporter mouse model expressing SpCas9 endogenously. This work indicates that neonatal AAV-guide recipients show higher rates of HSC gene editing in comparison to adult counterparts. Moreover, work described herein demonstrates that the use of self-complementary AAV vectors (as opposed to the standard single-stranded AAV vectors) results in significantly higher editing rates, e.g., in HSCs. Further functional characterization of edited HSCs indicates intact engraftment of gene-edited HSCs upon transplantation. These efforts aim to identify desirable conditions/vector design for the application of in vivo gene editing of HSCs in monogenic blood diseases.

The present invention provides compositions for and methods of genetically engineering hematopoietic stem and progenitor cells (HSPCs), such as hematopoietic stem cells (HSCs), a hematopoietic progenitor cell (HPCs), in vivo.

In one aspect, the disclosure features a method for treating a blood disorder in a subject the method including administering to the subject a nucleic acid molecule including a nucleotide sequence encoding two to six guide RNAs (gRNAs), wherein: (a) the nucleic acid molecule is delivered into a hematopoietic stem cell (HSC), a hematopoietic progenitor cell (HPC), or a population of hematopoietic stem and progenitor cells (HSPCs); and (b) each gRNA is capable of directing a sequence-targeting nuclease to a target locus in the genome of HSC, HPC, or HSPCs.

In another aspect, the disclosure features a method for treating a blood disorder in a subject the method including administering to the subject a nucleic acid molecule including a nucleotide sequence encoding: (a) two to six gRNAs; and (b) a sequence-targeting nuclease, wherein the nucleic acid molecule is delivered into an HSC, an HPC, or a population of HSPCs and each gRNA is capable of directing the sequence-targeting nuclease to a target locus in the genome of HSC, HPC, or HSPCs.

In yet another aspect, the disclosure features a method for removing a suppressor element in a subject the method including administering to the subject a nucleic acid molecule including a nucleotide sequence encoding two to six gRNAs, wherein: (a) the nucleic acid molecule is delivered into an HSC, an HPC, or a population of HSPCs; and (b) each gRNA is capable of directing a sequence-targeting nuclease to a target locus in the genome of HSC, HPC, or HSPCs.

In yet another aspect, the disclosure features a method for removing a suppressor element in a subject the method including administering to the subject a nucleic acid molecule including a nucleotide sequence encoding: (a) two to six guide RNAs; and (b) a sequence-targeting nuclease, wherein the nucleic acid molecule is delivered into an HSC, an HPC, or a population of HSPCs and each gRNA is capable of directing the sequence-targeting nuclease to target locus in the genome of HSC, HPC, or HSPCs.

In some aspects, the population of HPSCs include a hematopoietic stem cell (HSC) and/or a hematopoietic progenitor cell (HPC).

In some aspects, the nucleic acid molecule is an mRNA molecule, a plasmid, or a viral vector.

In some aspects, the viral vector is an adeno-associated virus (AAV). In some aspects, the AAV is a self-complementary AAV (scAAV).

In some aspects, the scAAV is about 1 kilobase (kb) to about 3.3 kb in length (e.g., about 1 kb to about 2.5 kb, about 1.5 kb to about 3 kb, about 2 kb to about 2.5 kb, about 2 kb to about 2.3 kb, about 2.5 kb to about 3 kb, or about 2.7 kb to about 3.3 kb in length). In some aspects, the scAAV is about 1.8 kb to about 2.1 kb in length (e.g., about 1.8 kb, about 1.9 kb, about 2 kb, or about 2.1 kb in length).

In some aspects, the nucleotide sequence encodes two gRNAs. In some aspects, the nucleotide sequence encodes three gRNAs. In some aspects, the nucleotide sequence encodes four gRNAs. In some aspects, the nucleotide sequence encodes five gRNAs. In some aspects, the nucleotide sequence encodes six gRNAs.

In some aspects, the gRNAs are operably linked to a different promoter.

In some aspects, the promoter is a constitutive promoter. In some aspects, the promoter is a ubiquitous promoter. In some aspects, the promoter is a human promoter, a viral promoter, or a bacterial promoter.

In some aspects, the promoter is a cytomegalovirus (CMV) promoter, a retrovirus promoter, a simian virus promoter, a papilloma virus promoter, a herpes virus promoter, an elongation factor-1 alpha (EF1α) promoter, a ubiquitin promoter, a globin promoter, an actin globin promoter, a phosphoglycerate kinase (PGK) globin promoter, a CAG promoter, a U6 promoter, a 7SK promoter, or an H1 promoter. In some aspects, the promoter is the U6 promoter, the H1 promoter, or the 7SK promoter.

In some aspects, the nucleic acid further includes a nucleotide sequence encoding the sequence-targeting nuclease.

In some aspects, the method further includes administering to the subject a second nucleic acid molecule including a nucleotide sequence encoding the sequence-targeting nuclease.

In some aspects, the method further includes administering to the subject a polypeptide of the sequence-targeting nuclease, wherein the polypeptide in packaged into a liposome or lipid nanoparticle (LNP).

In some aspects, the sequence-targeting nuclease is a Clustered Regularly Interspaced Short Palindromic Repeats (CRISPR)-associated (Cas) nuclease. In some aspects, the Cas nuclease is about 400 amino acids to about 2000 amino acids in size. In some aspects, the Cas nuclease is about 550 amino acids to about 1120 amino acids in size.

In some aspects, the Cas nuclease is a Cas9 nuclease, a Cas12a nuclease, or a Cas12f nuclease. In some aspects, the Cas9 nuclease is from Staphylococcus auricularis (Sa) or Streptococcus pyogenes (Sp). In some aspects, the Cas12a or Cas12f nuclease is from Lachnospiraceae bacterium (Lb).

In some aspects, the nucleic acid molecule is administered to the subject intravenously. In some aspects, the nucleic acid molecule is administered by intravenous infusion.

In some aspects, the blood disorder is a hemoglobinopathy, a primary immunodeficiency, a viral infection in the blood, a cytopenia, or a storage or metabolic disorder.

In some aspects, the hemoglobinopathy is sickle cell disease (SCD) or beta thalassemia (β-thalassemia). In some aspects, the primary immunodeficiency is X-linked severe combined immunodeficiency (X-SCID), adenosine deaminase severe combined immunodeficiency (ADA-SCID), Wiskott-Aldrich syndrome (WAS), or chronic granulomatous disease (CGD). In some aspects, the viral infection is a human immunodeficiency virus (HIV), human herpesvirus (HHV), or cytomegalovirus (CMV) infection in the blood. In some aspects, the cytopenia is Fanconi anemia (FA) or Shwachman-Diamond syndrome (SDS). In some aspects, the storage or metabolic disorder is Gaucher disease or X-linked adrenoleukodystrophy (X-ALD).

In some aspects, after administration of the nucleic acid molecule, the target locus in the genome of the HSPCs (e.g., the HSC or HPC) is disrupted by the nuclease activity of the sequence-targeting nuclease. In some aspects, the target locus is disrupted by an insertion or a deletion of a nucleotide in the target locus. In some aspects, the target locus is excised from the genome by nuclease activity at both the 5′ and 3′ end of the target locus.

In some aspects, the target locus is an intron, an exon, or a regulatory DNA element. In some aspects, the DNA regulatory element is an enhancer region, a suppressor region, or an insulator region. In some aspects, the suppressor region is repressing expression of fetal hemoglobin (HbF) in the subject. In some aspects, the suppressor region is a binding site of B-cell lymphoma/leukemia 11 (BCL11A).

In yet another aspect, the disclosure features a composition including: (a) a nucleic acid molecule including a nucleotide sequence encoding at least two (e.g., two, three, four, five, or six) gRNAs; and (b) a pharmaceutically acceptable carrier, excipient, or diluent, wherein each gRNA is capable of directing a sequence-targeting nuclease to a target locus in the genome of an HSC, an HPC, or a population of HSPCs.

In yet another aspect, the disclosure features a composition including a nucleic acid molecule including a nucleotide sequence encoding: (a) at least two (e.g., two, three, four, five, or six) gRNAs; (b) a sequence-targeting nuclease; and (c) a pharmaceutically acceptable carrier, excipient, or diluent, wherein each gRNA is capable of directing the sequence-targeting nuclease to a target locus in the genome of an HSC, an HPC, or a population of HSPCs.

In yet another aspect, the disclosure features a composition including: (a) a nucleic acid molecule including a nucleotide sequence encoding at least (e.g., two, three, four, five, or six) two gRNAs; (b) a sequence-targeting nuclease; and (c) a pharmaceutically acceptable carrier, excipient, or diluent, wherein each gRNA is capable of directing the sequence-targeting nuclease to a target locus in the genome of an HSC, an HPC, or a population of HSPCs.

In some aspects, the population of HPSCs include an HSC and/or an HPC.

In some aspects, the nucleic acid molecule is an mRNA molecule, a plasmid, or a viral vector.

In some aspects, the viral vector is an AAV. In some aspects, the AAV is an scAAV.

In some aspects, the scAAV is about 1 kb to about 3.3 kb in length (e.g., about 1 kb to about 2.5 kb, about 1.5 kb to about 3 kb, about 2 kb to about 2.5 kb, about 2 kb to about 2.3 kb, about 2.5 kb to about 3 kb, or about 2.7 kb to about 3.3 kb in length). In some aspects, the scAAV is about 1.8 kb to about 2.1 kb in length (e.g., about 1.8 kb, about 1.9 kb, about 2 kb, or about 2.1 kb in length).

In some aspects, the nucleotide sequence encodes two gRNAs. In some aspects, the nucleotide sequence encodes three gRNAs. In some aspects, the nucleotide sequence encodes four gRNAs. In some aspects, the nucleotide sequence encodes five gRNAs. In some aspects, the nucleotide sequence encodes six gRNAs.

In some aspects, each of the gRNAs are operably linked to a different promoter. In some aspects, the promoter is a constitutive promoter. In some aspects, the promoter is a ubiquitous promoter.

In some aspects, the promoter is a human promoter, a viral promoter, or a bacterial promoter.

In some aspects, the promoter is a CMV promoter, a retrovirus promoter, a simian virus promoter, a papilloma virus promoter, a herpes virus promoter, an EF1α promoter, a ubiquitin promoter, a globin promoter, an actin globin promoter, a PGK globin promoter, a CAG promoter, a U6 promoter, a 7SK promoter, or an H1 promoter. In some aspects, the promoter is the U6 promoter, the H1 promoter, or the 7SK promoter.

In some aspects, the nucleic acid molecule further includes a nucleotide sequence encoding the sequence-targeting nuclease.

In some aspects, the composition further includes a second nucleic acid molecule having a nucleotide sequence encoding the sequence-targeting nuclease.

In some aspects, the sequence-targeting nuclease is packaged in a liposome or LNP as a polypeptide.

In some aspects, the sequence-targeting nuclease is a Cas nuclease. In some aspects, the Cas nuclease is about 400 amino acids to about 2000 amino acids in size. In some aspects, the Cas nuclease is about 550 amino acids to about 1120 amino acids in size.

In some aspects, the Cas nuclease is a Cas9 nuclease, a Cas12a nuclease, or a Cas12f nuclease. In some aspects, the Cas9 nuclease is from Sa or Sp. In some aspects, the Cas12a or Cas12f nuclease is from Lb.

In some aspects, the composition is formulated for intravenous administration (e.g., intravenous infusion).

In some aspects, the blood disorder is a hemoglobinopathy, a primary immunodeficiency, a viral infection in the blood, a cytopenia, or a storage or metabolic disorder.

In some aspects, the hemoglobinopathy is SCD or beta thalassemia β-thalassemia. In some aspects, the primary immunodeficiency is X-SCID, ADA-SCID, WAS, or CGD. In some aspects, the viral infection is an HIV, HHV, or CMV infection in the blood. In some aspects, the cytopenia is FA or SDS. In some aspects, the storage or metabolic disorder is Gaucher disease or X-ALD.

In yet another aspect, the disclosure features a kit for treating a blood disorder in a subject including: (a) a first nucleic acid molecule having a nucleotide sequence encoding at least two (e.g., two, three, four, five, or six) gRNAs, wherein each gRNA is capable of directing a sequence-targeting nuclease to a target locus in a genome of an HSC, an HPC, or a population of HSPCs and optionally a pharmaceutically acceptable carrier, excipient, or diluent; and (b) a sequence-targeting nuclease or a second nucleic acid molecule encoding the sequence-targeting nuclease, and optionally a pharmaceutically acceptable carrier, excipient, or diluent.

In some aspects, the population of HPSCs include an HSC and/or an HPC.

In some aspects, the first or second nucleic acid molecule is an mRNA molecule, a plasmid, or a viral vector.

In some aspects, the viral vector is an AAV. In some aspects, the AAV is an scAAV.

In some aspects, the scAAV is about 1 kb to about 3.3 kb in length (e.g., about 1 kb to about 2.5 kb, about 1.5 kb to about 3 kb, about 2 kb to about 2.5 kb, about 2 kb to about 2.3 kb, about 2.5 kb to about 3 kb, or about 2.7 kb to about 3.3 kb in length). In some aspects, the scAAV is about 1.8 kb to about 2.1 kb in length (e.g., about 1.8 kb, about 1.9 kb, about 2 kb, or about 2.1 kb in length).

In some aspects, the nucleotide sequence encodes two gRNAs. In some aspects, the nucleotide sequence encodes three gRNAs. In some aspects, the nucleotide sequence encodes four gRNAs. In some aspects, the nucleotide sequence encodes five gRNAs. In some aspects, the nucleotide sequence encodes six gRNAs.

In some aspects, each of the gRNAs are operably linked to a different promoter. In some aspects, the promoter is a constitutive promoter. In some aspects, the promoter is a ubiquitous promoter.

In some aspects, the promoter is a human promoter, a viral promoter, or a bacterial promoter.

In some aspects, the promoter is a CMV promoter, a retrovirus promoter, a simian virus promoter, a papilloma virus promoter, a herpes virus promoter, an EF1α promoter, a ubiquitin promoter, a globin promoter, an actin globin promoter, a PGK globin promoter, a CAG promoter, a U6 promoter, a 7SK promoter, or an H1 promoter. In some aspects, the promoter is the U6 promoter, the H1 promoter, or the 7SK promoter.

In some aspects, the kit include the sequence-targeting nuclease as a polypeptide.

In some aspects, the kit includes the second nucleic acid molecule.

In some aspects, the polypeptide is packaged into a liposome or LNP.

In some aspects, the sequence-targeting nuclease is a Cas nuclease. In some aspects, the Cas nuclease is about 400 amino acids to about 2000 amino acids in size. In some aspects, the Cas nuclease is about 550 amino acids to about 1120 amino acids in size.

In some aspects, the Cas nuclease is a Cas9 nuclease, a Cas12a nuclease, or a Cas12f nuclease. In some aspects, the Cas9 nuclease is from Sa or Sp. In some aspects, the Cas12a or Cas12f nuclease is from Lb.

In some aspects, the components are formulated for intravenous administration (e.g., intravenous infusion).

In some aspects, the blood disorder is a hemoglobinopathy, a primary immunodeficiency, a viral infection in the blood, a cytopenia, or a storage or metabolic disorder.

In some aspects, the hemoglobinopathy is SCD or beta thalassemia β-thalassemia. In some aspects, the primary immunodeficiency is X-SCID, ADA-SCID, WAS, or CGD. In some aspects, the viral infection is an HIV, HHV, or CMV infection in the blood. In some aspects, the cytopenia is FA or SDS. In some aspects, the storage or metabolic disorder is Gaucher disease or X-ALD.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A and FIG. 1B show that systemic injection of AAV8-CRISPR disrupts Tet2 in peripheral blood cells. FIG. 1A shows that systemic injection of self-complementary (sc) AAV8-TRISPR, encoding three Tet2-targeting gRNAs, into adult mice harboring a blood lineage-restricted transgene encoding SpCas9 produces detectable disruption (insertions and deletions, indels) in the Tet2 gene within 4 weeks. FIG. 1B demonstrates that Tet2-mutant clones are maintained and expand as expected in the peripheral blood of AAV-injected animals.

FIG. 2A-FIG. 2E demonstrate transduction of both human and mouse bone marrow cells in adult humanized mice (mice transplanted with human CD34+ progenitors so that they have circulating human blood cells and HSCs) by systemically administered self-complementary AAV8 carrying a CBh promoter-GFP cargo (to mark transduced cells with green fluorescence). GFP+ bone marrow cells can be seen in the AAV-injected but not control (non-injected and FFB vehicle injected) humanized mice. GFP+ cells include human cells and mouse cells and mature lineages and lin-progenitors. FIG. 2A shows all live cells, both human and mouse; FIG. 2B shows only mouse cells (DAPI−, hHLA−, hCD45−, mCD45.1+); FIG. 2C shows only human myeloid cells (DAPI−, hHLA+, hCD45+, mCD45.1−, hCD33+, hMyeloidLin+); FIG. 2D shows only human lymphoid cells (DAPI−, hHLA+, hCD45+, mCD45.1−, hCD33−, hLymphoidLin+); and FIG. 2E shows only human progenitor cells. (DAPI−, hHLA+, hCD45+, mCD45.1−, hCD34+, hMyeloidLin−, hLymphoidLin−, hCD235a−).

FIG. 3 provides a summary of the data above, showing GFP transduction by scAAV8-Gfp in various human blood cell lineages (including progenitors that contain HSCs) and mouse blood cells.

FIG. 4 shows the results of work in which neonatal Sp-Cas9-expressing Ai9 mice (harboring a Lox-STOP-Lox allele upstream of tdTomato and transgenic expression of a SpCas9-GFP cassette) were injected with the indicated dose of either single stranded AAV (ssAAV) encoding 1 copy of the gRNA targeting upstream and 1 of the gRNA targeting downstream of the STOP cassette (single) or with self-complementary AAV (scAAV) encoding 2 copies of the gRNA targeting upstream and 2 copies of the gRNA targeting downstream of the STOP cassette. One month later, CD150+CD48-hematopoietic stem cells were identified by flow cytometry and assessed for tdTomato expression (an indicator of successful CRISPR gene editing at the Ai9 locus). Editing rates were substantially higher with the scAAV+2x gRNA format and reached 4% of HSCs at the highest vg dose. Editing rates with the ssAAV+1x gRNA format and editing rates with lower doses of scAAV+2x gRNAs typically ranged from 0.2-1%. AAV serotype=AAV8.

FIG. 5 is a schematic illustrating the experimental strategy described in Example 1. The top schematic depicts transgenic SpCas9-eGFP mice that exhibit constitutive and widespread expression of Streptococcus pyogenes Cas9 (SpCas9) and eGFP expressed from the Rosa26 locus under the control of a CAG promoter. Ai9 mice carry a CMV-lox-Stop-lox (LSL)-TdTomato transgene, also knocked into Rosa26. CRISPR-mediated cutting 5′ and 3′ of the Ai9 LSL results in excision of Stop cassette and expression of TdTomato. The bottom schematic depicts adeno associated virus (AAV) vectors utilized in the Ai9;Cas9-eGFP model. Three vector configurations were used in the studies: a) single-stranded (ss)-AAV-1xgRNAs in which one copy of each “Left” and “Right” guide RNAs (gRNAs), targeting 5′ and 3′ of the Ai9 LSL, respectively, is driven by human U6 promoters in single-stranded AAVs, b) self-complementary (sc)-AAV-1xgRNAs which also has one copy of each gRNA but is in a self-complementary format, c) scAAV-2xgRNAs which is self-complementary with two copies of each gRNA. Promoters used for each gRNA are listed.

FIG. 6A is a schematic illustrating mice injected through retro-orbital route for systemic delivery of AAV. Initially, two doses of AAV were used: 5×1011 viral genomes (vg)/animal and 5×1012 vg/animal.

FIG. 6B shows a representative analysis of bone marrow (BM) HSCs by flow cytometry showing progressing exclusion of non-relevant cell populations based on marker expression. HSCs are defined as Lineage−Kit+Sca+CD150+CD48−.

FIG. 7A is a graph depicting the percent of TdTomato-positive (% TdTomato+) HSCs in Ai9;Cas9 mice injected with vehicle control or 5×1011 vg/animal and 5×1012 vg/animal of AAV.

FIG. 7B is a graph depicting % TdTomato+ HSCs in Ai9;Cas9 mice injected with SSAAV-1xgRNA or scAAV-1xgRNA (e.g., a ssAAV versus scAAV configuration).

FIG. 7C is a graph depicting % TdTomato+ HSCs in Ai9;Cas9 mice injected with scAAV-1xgRNA or scAAV-2xgRNA (e.g., a one-copy versus two copy comparison).

FIG. 8A is a schematic illustrating the experimental design for testing HSC gene modification in Ai9;Cas9 newborn-injected animals. In brief, newborn animals were systemically injected with the vehicle or vector through the facial vein. Animals were analyzed about 8 weeks post injection.

FIG. 8B is a graph depicting % TdTomato+ BM HSCs in animals injected as newborns with vehicle or four different doses of ssAAV-1xgRNA.

FIG. 8C is a graph depicting % TdTomato+ BM HSCs in animals injected as newborns with vehicle or scAAV-2xgRNA.

FIG. 9A is a schematic illustrating an HSC gene modification strategy in a sickle cell disease (SCD) animal model (e.g., SCD;Cas9). In brief, transgenic animals expressing endogenous SpCas9-eGFP as well human globin genes were injected with vehicle or 4×1012 of scAAV-TRISPR.HBG.guide to target and disrupt the B-cell lymphoma/leukemia 11 (BCL11A) binding motif in the HBG promoter. Thirteen weeks post injection animals were assayed for HSC gene modification rates.

FIG. 9B is a graph depicting percent modified reads (signifying insertions or deletions in the HBG promoter) detected in the whole blood of mice at 4 weeks post injection. The mouse harbors human hemoglobin alpha (HBA), hemoglobin beta (HBB), and hemoglobin gamma (HBG) genes. A/A are homozygotes for wild-type HBB whereas S/S animals carry two copies of sickle HBB.

FIG. 9C is a graph depicting percent modified reads (signifying insertions or deletions in the HBG promoter) detected in the whole blood of mice at 9 weeks post injection.

FIG. 9D is a graph depicting percent modified reads (signifying insertions or deletions in the HBG promoter) detected in the whole blood of mice at 13 weeks post injection.

FIG. 9E is a graph depicting percent modified reads (signifying insertions or deletions) in the HBG promoter target in HSCs. The data shows successful in vivo gene modification of HSCs in a mouse model (“SCD;Cas9”) of SCD.

### DETAILED DESCRIPTION OF THE INVENTION

Described herein are methods for precise, targeted gene correction and/or replacement, allowing for direct manipulation of cells, e.g., stem cell genomes, therapeutically and experimentally, without the need to isolate, expand or transplant these cells. The teachings and methods of PCT/US2019/030748 in this regard are incorporated herein by reference in their entirety. Disclosed herein are methods of treating genetic blood disorders in a subject by way of genetically editing a population of hematopoietic stem and progenitor cells (HSPCs) in vivo (also referred to as in situ). As used herein, HSPCs may include hematopoietic stem cells (HSCs) and/or hematopoietic progenitor cells (HPCs). The methods utilize the delivery of guide RNAs (gRNAs) and sequence-targeting nucleases to HSPCs (e.g., HSCs and/or HPCs) in vivo. Advantageously, the methods of treatment described herein may utilize, e.g., a self-complementary adeno-associated viral vector (scAAV) containing at least two gRNAs, resulting in increased transduction and gene editing efficiencies of HSPCs (e.g., HSCs and/or HPCs) in vivo. The methods of treatment described herein avoid the complications of extracting HSPCs (e.g., HSCs and/or HPCs) from a subject in need of treatment. With the methods described herein, viral vectors (e.g., scAAVs) can be administered directly into, e.g., via the blood stream, the subject to produce a therapeutic effect, thus circumventing the need to perform ex vivo HSC modification and HSC transplantation (HSCT). Furthermore, the compositions and methods described herein result in increased transfection at therapeutically relevant levels.

### Selected Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context (except where such number would exceed 100% of a possible value).

As used herein, “administration” refers to providing or giving a subject a therapeutic agent by any effective route. Exemplary routes of administration are described herein.

As used herein, the term “CRISPR-Cas” refers to a complex/system that can include a crRNA, a crRNA and a trcrRNA, or a gRNA bound to a Cas enzyme.

As used herein, the term a “disorder” is any condition that would benefit from treatment including, but not limited to, chronic and acute disorders or diseases including those pathological conditions which predispose a mammal to the disorder in question.

As used herein, the terms “effective amount,” “therapeutically effective amount,” and a “sufficient amount” of a composition described herein refer to a quantity sufficient to, when administered to the subject, including a mammal, for example a human, effect beneficial or desired results, including clinical results, and, as such, an “effective amount” or synonym thereto depends upon the context in which it is being applied

As used herein, the term guide RNA (gRNA) refers to a single RNA sequence capable of directing RNA-guided endonuclease-mediated cleavage of target nucleic acid molecule. The crRNA region of the gRNA is a customizable component that enables specificity in every CRISPR reaction. A gRNA can include any polynucleotide sequence having sufficient complementarity with a target sequence to hybridize with the target sequence and to direct sequence-specific binding of a CRISPR-Cas complex to the target sequence. A gRNA may contain a crRNA sequence, but not a trcrRNA sequence. Alternatively, a gRNA may contain both a crRNA sequence and a trcrRNA sequence.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” or “in situ” refers to genome editing events that occur within an organism (e.g., animal, plant, or microbe or cell or tissue thereof).

As used herein, a “subject” means a human or animal (e.g., a primate). Usually, the animal is a vertebrate such as a primate, rodent, domestic animal, or game animal. Primates include chimpanzees, cynomologous monkeys, spider monkeys, and macaques, e.g., Rhesus. Rodents include mice, rats, woodchucks, ferrets, rabbits, and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish, and salmon.

As used herein, the term “target sequence” refers to a nucleic acid sequence that is recognized by a gRNA or crRNA sequence in a CRISPR-Cas complex. The gRNA or crRNA sequence contains one or more spacer sequences that have complementarity to the target sequence(s) of interest. The spacer sequence of a gRNA or crRNA may be 50%, 60%, 75%, 80%, 85%, 90%, 95%, 97.5%, 98%, 99%, or more complementary to the target sequence of interest. Optimal alignment can be determined with the use of any suitable algorithm for aligning sequences. Full complementarity is not necessarily required, provided there is sufficient complementarity to cause hybridization and cis-endonuclease activation of the CRISPR-Cas complex. A target sequence can include any polynucleotide, such as DNA or RNA polynucleotides. A target sequence of interest may be located in the nucleus or cytoplasm of a cell such as, for example, within an organelle of a eukaryotic cell, such as a mitochondrion or a chloroplast, or it can be exogenous to a host cell.

As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages.

In some embodiments, the invention relates to methods of modifying the genome of a target cell in vivo in a subject, comprising contacting the cell with one or more scAAV, wherein the one or more scAAV transduce a nucleic acid sequence encoding a sequence-targeting nuclease into the target cell. The target cell can be any biologically or therapeutically relevant cell, and the scAAV can be selected or optimized to specifically target the target cell. In other embodiments, the scAAV transduce one or more guide RNAs (gRNAs) into the target cell. In some embodiments multiple gRNAs specific for the same target site are transduced into the target cell.

As used herein, a “subject” means a human or animal (e.g., a primate). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein. Preferably, the subject is a mammal. The mammal can be a human, non-human primate, mouse, rat, dog, cat, horse, or cow, but are not limited to these examples. A subject can be male or female. A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments, a human subject is between newborn and 6 months old. In some embodiments, a human subject is between 6 and 24 months old. In some embodiments, a human subject is between 2 and 6, 6 and 12, or 12 and 18 years old. In some embodiments a human subject is between 18 and 30, 30 and 50, 50 and 80, or greater than 80 years old. In some embodiments, the subject is at least about 5, 10, 20, 30, 40, 50, 60, 65, 70, 75, 80, 85, or 90 years of age. In some embodiments, the subject is less than about 5, 10, 20, 30, 40, 50, 60, 65, 70, 75, 80, 85, or 90 years of age. In some embodiments, a subject is an adult. For purposes hereof a human at least 18 years of age is considered an adult. In some embodiments, the subject is a juvenile (e.g., less than about 18, 12 or 6 years of age for a human subject). In some embodiments, the subject is not a juvenile (e.g., less than about 18, 12 or 6 years of age for a human subject). In some embodiments a subject is an embryo. In some embodiments a subject is a fetus. In certain embodiments an agent is administered to a pregnant female in order to treat or cause a biological effect on an embryo or fetus in utero.

As used herein, “contacting” a cell with one or more viruses can comprise administration of the virus systemically (e.g., intravenously) or locally (e.g., intramuscular injection) into the subject. Alternatively, other routes of administration may be selected (e.g., oral, inhalation, intranasal, intratracheal, intraarterial, intraocular, intravenous, intramuscular, and other parental routes). The method of contacting is not limited and may be any suitable method available in the art.

In some embodiments, virus compositions can be formulated in dosage units to contain an amount of replication-defective virus that is in the range of about 1.0×10⁹ GC to about 1.0'1015 GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

In some embodiments, at least about 0.1%, 0.5%, 1%, 2%, 3%, 5%, 10%, 15%, 20%, 25%, 30%, 35%, 40%, 45%, 50% or more of the genomes of the target cells or a subset thereof are modified. In some embodiments, at least about 0.1%, 0.5%, 1%, 2%, 3%, 5%, 10%, 15%, 20%, 25%, 30%, 35%, 40%, 45%, 50% or more of the genome of the target cells or a subset thereof are modified via homologous recombination (e.g., a genomic sequence is replaced or inserted via homologous recombination). In some embodiments, at least about 40% or more of the genome of the target cells or a subset thereof are modified via homologous recombination (e.g., a genomic sequence is replaced or inserted via homologous recombination). In some embodiments, at least 1% of target cells in the subject are modified to comprise an insertion of a nucleotide sequence corresponding to a nucleotide sequence of the donor template. In some embodiments, at least 1%, 2%, 3%, 5%, 10%, 15%, 20%, 25%, 30%, 35%, 40%, 45%, 50% or more of the target cells in the subject are modified to comprise an insertion of a nucleotide sequence corresponding to a nucleotide sequence of the donor template. In some embodiments, the modification comprises a modification of at least one allele. In some embodiments, the modification comprises modification of both alleles.

Suitable viruses for use in the methods disclosed throughout the specification include, e.g., adenoviruses, adeno-associated viruses, retroviruses (e.g., lentiviruses), vaccinia virus and other poxviruses, herpesviruses (e.g., herpes simplex virus), and others. The virus may or may not contain sufficient viral genetic information for production of infectious virus when introduced into host cells, i.e., viral vectors may be replication-competent or replication-defective.

In some embodiments, the virus is adeno-associated virus. Adeno-associated virus (AAV) is a small (20 nm) replication-defective, nonenveloped virus. The AAV genome a single-stranded DNA (ssDNA) about 4.7 kilobase long. The genome comprises inverted terminal repeats (ITRs) at both ends of the DNA strand, and two open reading frames (ORFs): rep and cap. The AAV genome integrates most frequently into a particular site on chromosome 19. Random incorporations into the genome take place with a negligible frequency. The integrative capacity may be eliminated by removing at least part of the rep ORF from the vector resulting in vectors that remain episomal and provide sustained expression at least in non-dividing cells. To use AAV as a gene transfer vector, a nucleic acid comprising a nucleic acid sequence encoding a desired protein or RNA, e.g., encoding a polypeptide or RNA that inhibits ATPIF1, operably linked to a promoter, is inserted between the inverted terminal repeats (ITR) of the AAV genome. Adeno-associated viruses (AAV) and their use as vectors, e.g., for gene therapy, are also discussed in Snyder, RO and Moullier, P., Adeno-Associated Virus Methods and Protocols, Methods in Molecular Biology, Vol. 807. Humana Press, 2011.

In some embodiments, the AAV is AAV serotype 6, 8, 9, 10 or Anc80 (disclosed in WO2015054653, incorporated herein by reference). In some embodiments, the AAV serotype is AAV serotype 2. Any AAV serotype, or modified AAV serotype, may be used as appropriate and is not limited.

Another suitable AAV may be, e.g., rhIO [see, e.g., WO 2003/042397]. Still other AAV sources may include, e.g., AAV9 [see, e.g., U.S. Pat. No. 7,906,111; US 2011-0236353-A1], and/or hu37 [see, e.g., U.S. Pat. No. 7,906,111; US 2011-0236353-A1], AAV1, AAV2, AAV3, AAV4, AAV5, AAV6, AAV6.2, AAV7, AAV8, [see, e.g., U.S. Pat. Nos. 7,790,449; 7,282,199] and others. See, e.g., WO 2003/042397; WO 2005/033321, WO 2006/110689; U.S. Pat. Nos. 7,790,449; 7,282,199; and 7,588,772 B2 for sequences of these and other suitable AAV, as well as for methods for generating AAV vectors. Still other AAV may be selected, optionally taking into consideration tissue preferences of the selected AAV capsid. A recombinant AAV vector (AAV viral particle) may comprise, packaged within an AAV capsid, a nucleic acid molecule containing a 5′AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ITR. A shortened version of the 5′ ITR, termed AITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

Additional useful vectors include self-complementary adeno-associated virus (scAAV), a viral vector engineered from the naturally occurring adeno-associated virus (AAV). This synthetic progeny of rAAV is termed “self-complementary” because the coding region has been designed to form an intra-molecular double-stranded DNA template. A rate-limiting step for the standard AAV genome involves the second-strand synthesis since the typical AAV genome is a single-stranded DNA template. However, this is not the case for scAAV genomes. Upon infection, rather than waiting for cell-mediated synthesis of the second strand, the two complementary halves of scAAV will associate to form one double stranded DNA (dsDNA) unit that is ready for immediate replication and transcription. Additional advantages of scAAV may include increased and prolonged transgene expression in vitro and in vivo, as well as higher in vivo DNA stability and more effective circularization.

A single-stranded AAV viral vector may also be used. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV-the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al, 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of which is incorporated herein by reference in its entirety: U.S. Pat. Nos.5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In another embodiment, other viral vectors may be used, including integrating viruses, e.g., herpesvirus or lentivirus, although other viruses may be selected. Suitably, where one of these other vectors is generated, it is produced as a replication-defective viral vector. A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production.

The one or more viruses may contain a promoter capable of directing expression (e.g., expression of a sequence-targeting nuclease, donor template, and/or one or more gRNAs) in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, the promoter is selected from a CMV promoter, U6 promoter, an H1 promoter, a constitutive promoter, and a ubiquitous promoter. In some embodiments, the promoter directs expression in a particular cell type. For example, a muscle precursor cell specific promoter.

In some embodiments of each of the methods disclosed herein, a suitable tissue specific promoter can be obtained by a person of ordinary skill in the art from the tissue specific promoters set forth in “TiProD: Tissue specific promoter Database” available on the world-wide web at tiprod.bioinf.med.uni-goettingen.de/.

The sequence-targeting nucleases that can be used in the methods disclosed herein are not limited and may be any sequence-targeting nucleases disclosed herein. In some embodiments, the sequence-targeting nuclease is a Zinc-Finger Nuclease (ZFN), a Transcription activator-like effector nuclease (TALEN), a Cas nuclease (e.g., Cas9 nuclease), or a functional fragment or functional variant thereof.

There are currently four main types of sequence-targeting nucleases (i.e., targetable nucleases, site specific nucleases) in use: zinc finger nucleases (ZFNs), transcription activator-like effector nucleases (TALENs), and RNA-guided nucleases (RGNs) such as the Cas proteins of the CRISPR/Cas Type II system, and engineered meganucleases. ZFNs and TALENs comprise the nuclease domain of the restriction enzyme FokI (or an engineered variant thereof) fused to a site-specific DNA binding domain (DBD) that is appropriately designed to target the protein to a selected DNA sequence. In the case of ZFNs, the DNA binding domain (DBD) comprises a zinc finger DBD. In the case of TALENs, the site-specific DBD is designed based on the DNA recognition code employed by transcription activator-like effectors (TALEs), a family of site-specific DNA binding proteins found in plant-pathogenic bacteria such as Xanthomonas species.

The Clustered Regularly Interspaced Short Palindromic Repeats (CRISPR) Type II system is a bacterial adaptive immune system that has been modified for use as an RNA-guided endonuclease technology for genome engineering. The bacterial system comprises two endogenous bacterial RNAs called crRNA and tracrRNA and a CRISPR-associated (Cas) nuclease, e.g., Cas9. The tracrRNA has partial complementarity to the crRNA and forms a complex with it. The Cas protein is guided to the target sequence by the crRNA/tracrRNA complex, which forms a RNA/DNA hybrid between the crRNA sequence and the complementary sequence in the target. For use in genome modification, the crRNA and tracrRNA components are often combined into a single chimeric guide RNA (sgRNA or gRNA) in which the targeting specificity of the crRNA and the properties of the tracrRNA are combined into a single transcript that localizes the Cas protein to the target sequence so that the Cas protein can cleave the DNA. The sgRNA often comprises an approximately 20 nucleotide guide sequence complementary or homologous to the desired target sequence followed by about 80 nt of hybrid crRNA/tracrRNA. One of ordinary skill in the art appreciates that the guide RNA need not be perfectly complementary or homologous to the target sequence. For example, in some embodiments it may have one or two mismatches. The genomic sequence which the gRNA hybridizes is typically flanked on one side by a Protospacer Adjacent Motif (PAM) sequence although one of ordinary skill in the art appreciates that certain Cas proteins may have a relaxed requirement for a PAM sequence. The PAM sequence is present in the genomic DNA but not in the sgRNA sequence. The Cas protein will be directed to any DNA sequence with the correct target sequence and PAM sequence. The PAM sequence varies depending on the species of bacteria from which the Cas protein was derived. Specific examples of Cas proteins include Casl, Cas2, Cas3, Cas4, Cas5, Cas6, Cas7, Cas8, Cas9 and Cas10. In some embodiments, the site specific nuclease comprises a Cas9 protein. For example, Cas9 from Streptococcus pyogenes (Sp), Neisseria meningitides, Staphylococcus aureus, Streptococcus thermophiles, or Treponema denticola may be used. The PAM sequences for these Cas9 proteins are NGG, NNNNGATT, NNAGAA, NAAAAC, respectively. In some embodiments, the Cas9 is from Staphylococcus aureus (saCas9).

A number of engineered variants of the site-specific nucleases have been developed and may be used in certain embodiments. For example, engineered variants of Cas9 and Fok1 are known in the art. Furthermore, it will be understood that a biologically active fragment or variant can be used. Other variations include the use of hybrid site specific nucleases. For example, in CRISPR RNA-guided FokI nucleases (RFNs) the FokI nuclease domain is fused to the amino-terminal end of a catalytically inactive Cas9 protein (dCas9) protein. RFNs act as dimers and utilize two guide RNAs (Tsai, QS, et al., Nat Biotechnol. 2014; 32(6): 569-576). Site-specific nucleases that produce a single-stranded DNA break are also of use for genome editing. Such nucleases, sometimes termed “nickases” can be generated by introducing a mutation (e.g., an alanine substitution) at key catalytic residues in one of the two nuclease domains of a site specific nuclease that comprises two nuclease domains (such as ZFNs, TALENs, and Cas proteins). Examples of such mutations include D10A, N863A, and H840A in SpCas9 or at homologous positions in other Cas9 proteins. A nick can stimulate HDR at low efficiency in some cell types. Two nickases, targeted to a pair of sequences that are near each other and on opposite strands can create a single-stranded break on each strand (“double nicking”), effectively generating a DSB, which can optionally be repaired by HDR using a donor DNA template (Ran, F. A. et al. Cell 154, 1380-1389 (2013). In some embodiments, the Cas protein is a SpCas9 variant. In some embodiments, the SpCas9 variant is a R661A/Q695A/Q926A triple variant or a N497A/R661A/Q695A/Q926A quadruple variant. See Kleinstiver et al., “High-fidelity CRISPR-Cas9 nucleases with no detectable genome-wide off-target effects,” Nature, Vol. 529, pp. 490-495 (and supplementary materials) (2016); incorporated herein by reference in its entirety. In some embodiments, the Cas protein is C2c1, a class 2 type V-B CRISPR-Cas protein. See Yang et al., “PAM-Dependent Target DNA Recognition and Cleavage by C2c1 CRISPR-Cas Endonuclease,” Cell, Vol. 167, pp. 1814-1828 (2016); incorporated herein by reference in its entirety. In some embodiments, the Cas protein is one described in US 20160319260 “Engineered CRISPR-Cas9 nucleases with Altered PAM Specificity” incorporated herein by reference.

The nucleic acid encoding the sequence-targeting nuclease should be sufficiently short to be included in the virus (e.g., AAV). In some embodiments, the nucleic acid encoding the sequence-targeting nuclease is less than 4.4. kb.

In some embodiments, the sequence-targeting nuclease has at least about 80%, 85%, 90%, 91%, 92%, 93%, 94%, 95%, 96%, 97%, 98% or 99% polypeptide sequence identity to a naturally occurring targetable nuclease.

In some embodiments, the one or more viruses comprise a first virus which transduces a nucleic acid sequence encoding a sequence-targeting nuclease and a donor template. In some embodiments, the one or more viruses comprise a first virus which transduces a nucleic acid sequence encoding a sequence-targeting nuclease, a donor template and one or more (e.g, one, two, three, four, etc.) gRNAs. In embodiments of the methods described herein wherein a single virus transduces the sequence-targeting nuclease, the donor template, and, optionally, one or more gRNAs a person of ordinary skill in the art can select a suitable virus capable of packaging the required nucleotide sequences. In some embodiments, the one or more viruses comprise a first virus which transduces a nucleic acid sequence encoding a sequence-targeting nuclease, and a second virus which transduces a donor template. In some embodiments, the one or more viruses comprise a first virus which transduces a nucleic acid sequence encoding a sequence-targeting nuclease, and a second virus which transduces a donor template and one or more (e.g, one, two, three, four, etc.) gRNAs. In some embodiments, the one or more viruses comprise a first virus which transduces a nucleic acid sequence encoding a sequence-targeting nuclease, and a second virus which transduces a donor template and two gRNAs. In some embodiment, the ratio of the first virus to the second virus is about 1:3 to about 1:100, inclusive of intervening ratios. For example, the ratio of the first virus to the second virus may be about 1:5 to about 1:50, or about 1:10, or about 1:20. Although not as preferred, the ratio may be 1:1 or there may be more second virus.

In some embodiments, the method comprises delivery of one or more components (e.g., nucleic acid encoding a sequence-targeting nuclease, a donor template, one or more gRNAs (e.g., two gRNAs)) mediated by non-viral constructs, e.g., “naked DNA”, “naked plasmid DNA”, RNA, and mRNA; coupled with various delivery compositions and nanoparticles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 201 1, 8 (3), pp 774-787; web publication: Mar. 21, 2011; WO2013/182683, WO 2010/053572 and WO 2012/170930, all of which are incorporated herein by reference.

The terms “decrease,” “reduce,” “reduced,” “reduction,” “decrease,” and “'inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The term “statistically significant” or “significantly” refers to statistical significance and generally means a “p” value greater than 0.05 (calculated by the relevant statistical test). Those skilled in the art will readily appreciate that the relevant statistical test for any particular experiment depends on the type of data being analyzed. Additional definitions are provided in the text of individual sections below.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10:0763766321); Kendrew et al. (eds.), Molecular Biology and Biotechnology: a Comprehensive Desk Reference, published by VCH Publishers, Inc., 1995 (ISBN 1-56081-569-8) and Cun-ent Protocols in Protein Sciences 2009, Wiley Intersciences, Coligan et al., eds.

As used herein, the terms “proteins” and “polypeptides” are used interchangeably to designate a series of amino acid residues connected to the other by peptide bonds between the alpha-amino and carboxy groups of adjacent residues. The terms “protein”, and “polypeptide” refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when refining to a gene product and fragments thereof.

Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double stranded DNA. Alternatively, it can be a single-stranded nucleic acid not derived from any double stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA. The nucleic acid molecule can be naturally occurring, as in genomic DNA, or it may be synthetic, i.e., prepared based upon human action, or may be a combination of the two. The nucleic acid molecule can also have certain modification such as 2′-deoxy, 2′-deoxy-2′fluoro, 2′-0-methyl, 2′-0-methoxyethyl (2′-0-MOE), 2′-0-aminopropyl (2′-0-AP), 2′-0-dimethylaminoethyl (2′-0-DMAOE), 2′-0-dimethylaminopropyl (2′-0-DMAP), 2′-0-dimethylaminoethyloxyethyl (2′-0-DMAEOE), or 2′-0--N-methylacetamido (2′-0-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

As used herein, “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state as compared to that expected in the absence of treatment.

The efficacy of a given treatment for a disorder or disease can be determined by the skilled clinician. However, a treatment is considered “effective treatment,” as the term is used herein, if any one or all of the signs or symptoms of a disorder are altered in a beneficial manner, other clinically accepted symptoms are improved or ameliorated, e.g., by at least 10% following treatment with an agent or composition as described herein. Efficacy can also be measured by a failure of an individual to worsen as assessed by hospitalization or need for medical interventions (i.e., progression of the disease is halted). Methods of measuring these indicators are known to those of skill in the art and/or described herein.

### Methods of Treatment

The present disclosure describes a method for modifying, in vivo, the genome of HSPCs (e.g., HSCs and/or HPCs) in a subject (e.g., human or mouse). The region of the genome to be modified (e.g., the target locus) may be associated with a blood disorder, such as a hemoglobinopathy (e.g., sickle cell disease (SCD) and beta thalassemia (β-thalassemia)), a primary immunodeficiency (e.g., X-linked severe combined immunodeficiency (X-SCID), adenosine deaminase severe combined immunodeficiency (ADA-SCID), Wiskott-Aldrich syndrome (WAS), and chronic granulomatous disease (CGD), a viral infection (e.g., a human immunodeficiency virus (HIV), human herpesvirus (HHV), or cytomegalovirus (CMV) infection), a cytopenia (e.g., Fanconi anemia (FA) and Shwachman-Diamond syndrome (SDS), or a storage or metabolic disorder (e.g., Gaucher disease and X-linked adrenoleukodystrophy (X-ALD). Modification of the target locus may result in treatment of the blood disorder or prevent the onset of symptoms of the blood disorder in the subject. For example, the method may include the step of administering to the subject (e.g., a subject having a genetic blood disorder) a virus (e.g., an AAV), wherein the virus transduces a nucleic acid having a nucleotide sequence encoding two or more gRNAs and (either in the same nucleic acid or a second nucleic acid from a second vector) a sequence-targeting nuclease into the HSPC (e.g., the HSC and/or HPC). Upon entry into the HSPC (e.g., the HSC and/or HPC), the transduced nucleic acid can express a plurality of gRNAs and sequence-targeting nucleases (e.g., Cas), which can form a plurality of nucleoprotein complexes in the HSPC. The gRNA of the nucleoprotein complex (e.g., a CRISPR-Cas complex) allows for specific binding of the complex to a target locus in the HSPC (e.g., the HSC and/or HPC) genome. Upon binding to the target locus, the nuclease can cleave (e.g., double stranded cis-cleavage) the target locus in the HSPC (e.g., the HSC and/or HPC) genome. Upon cleavage of the target locus, an insertion or deletion (indel) may naturally occur at the target locus, thereby disrupting the target locus. Alternatively, upon cleavage of two or more target loci, an entire region of a target locus can be excised from the HSPC (e.g., the HSC and/or HPC) genome, thereby disrupting the target locus. Such disruption of the HSPC (e.g., the HSC and/or HPC) target locus can treat, ameliorate, or prevent a blood disorder in the subject.

The efficacy of a given treatment for a disorder or disease can be determined by the skilled clinician. However, a treatment is considered “effective treatment,” as the term is used herein, if any one or all of the signs or symptoms of a disorder are altered in a beneficial manner, other clinically accepted symptoms are improved or ameliorated, e.g., by at least 10% following treatment with an agent or composition as described herein. Efficacy can also be measured by a failure of an individual to worsen as assessed by hospitalization or need for medical interventions (i.e., progression of the disease is halted). Methods of measuring these indicators are known to those of skill in the art and/or described herein.

### Administration

Provided herein is a method for treating a blood disorder in a subject by modifying the genome of a population of HSPCs (e.g., HSCs and/or HPCs) in a subject in vivo, the method including the step of administering to the subject a first nucleic acid molecule (e.g., an mRNA, plasmid, or viral vector), wherein the first nucleic acid molecule encodes two or more (e.g., two to six, e.g., two, three, four, five, or six) gRNAs in the population of HSPCs. Each gRNA may be capable of directing a sequence-targeting nuclease (which may be delivered separately, as described herein) to a target locus in the genome of an HSPC (e.g., HSC and/or HPC). The method may further include administering to the subject a second nucleic acid molecule (e.g., an mRNA, plasmid, or viral vector) encoding the sequence-targeting nuclease in a population of HSPCs (e.g., HSCs and/or HPCs); and modifying the genome of the population of HSPCs (e.g., HSCs and/or HPCs) with the encoded sequence-targeting nuclease. The first or second nucleic acid molecule is not limited and may be any described herein. When the first and second nucleic acid molecules are delivered into the HSPC (e.g., HSC and/or HPC), the encoded sequence-targeting nuclease and the two or more gRNAs are expressed. Once expressed, the sequence-targeting nuclease and the gRNA form a complex capable of modifying a target locus in the genome of the HSPC (e.g., HSCs and/or HPC).

Upon administration, the ratio of the first nucleic acid molecule (e.g., the mRNA, plasmid, or viral vector encoding two to six gRNAs) to the second nucleic acid molecule (e.g., the mRNA, plasmid, or viral vector encoding the sequence-targeting nuclease) may be about 1:1 to about 10:1, about 5:1 to about 50:1, or about 3:1 to about 100:1 (e.g., about 4:1 to about 20:1, about 10:1 to about 50:1, about 25:1 to about 75:1, about 50:1 to about 100:1). For example, the ratio of the first nucleic acid molecule to the second nucleic acid molecule may be about 5:1 to about 50:1 (e.g., about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, or 50:1), or about 10:1 (e.g., 9:1, 10:1, or 11:1), or about 20:1 (e.g., 18:1, 19:1, 20:1, 21:1, or 22:1).

In another embodiment, the methods provided herein include the step of administering to the subject a nucleic acid molecule (e.g., an mRNA, plasmid, or viral vector), wherein the nucleic acid molecule encodes a sequence-targeting nuclease and two or more (e.g., two, three, four, five, or six) gRNAs in a population of HSPCs (e.g., HSCs and/or HPCs). The method may further include the step of modifying the genome of the population of HSPCs (e.g., HSCs and/or HPCs) with the encoded sequence-targeting nuclease and gRNAs. The nucleic acid molecule is not limited and may be any described herein. When the nucleic acid molecule is delivered into the HSPC (e.g., HSC and/or HPC), the encoded sequence-targeting nuclease and two or more gRNAs are expressed. Once expressed, the sequence-targeting nuclease and a gRNA form a complex capable of modifying a target locus in the genome of the HSPC (e.g., HSC and/or HPC).

The nucleic acid molecule may be a viral vector (e.g., an scAAV) that is formulated in a viral particle, such as a viral particle derived from an AAV. AAVs are a small (20 nm) replication-defective, nonenveloped virus with a genome size of about 4.7 kilobase (kb) long. The genome contains inverted terminal repeats (ITRs) at both ends of the DNA strand, and two open reading frames (ORFs): rep and cap. The viral vector encoding the gRNA and/or the sequence-targeting nuclease is inserted between ITRs of the AAV genome. AAVs and their use as vectors, e.g., for gene therapy, are also discussed in Snyder, R O and Moullier, P., Adeno-Associated Virus Methods and Protocols, Methods in Molecular Biology, Vol. 807. Humana Press, 2011.

The method for treating the blood disorder in the subject may include the steps of administering to the subject a first viral (e.g., an AAV) particle which transduces a nucleic acid molecule (e.g., an scAAV) encoding two or more (e.g., two to six, e.g., two, three, four, five, or six) gRNAs in a population of HSPCs (e.g., HSCs and/or HPCs); and modifying the genome of the population of HSPCs (e.g., HSCs and/or HPCs) with a sequence-targeting nuclease. The method may further include administering to the subject a second viral (e.g., an AAV) particle which transduces a second nucleic acid molecule (e.g., an scAAV) encoding a sequence-targeting nuclease into a population of HSPCs (e.g., HSC and/or HPC). The first or second viral particle is not limited and may be derived from any virus described herein. When the first and second viral particle transduce their respective nucleic acid molecules into the HSPC (e.g., HS and/or HPC), the encoded sequence-targeting nuclease and the two or more gRNAs are expressed. Once expressed, the sequence-targeting nuclease and the gRNA form a complex capable of modifying a target locus in the genome of the HSPC (e.g., HSC and/or HPC).

Upon administration, the ratio of the first viral particle (e.g., the scAAV encoding two to six gRNAs) to the second viral particle (e.g., the scAAV encoding the sequence-targeting nuclease) may be about 3:1 to about 100:1 (e.g., about 4:1 to about 20:1, about 10:1 to about 50:1, about 25:1 to about 75:1, about 50:1 to about 100:1). For example, the ratio of the first viral particle to the second viral particle may be about 5:1 to about 50:1 (e.g., about 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, or 50:1), or about 10:1 (e.g., 9:1, 10:1, or 11:1), or about 20:1 (e.g., 18:1, 19:1, 20:1, 21:1, or 22:1).

In another embodiment, the methods provided herein include the steps of administering to the subject a viral (e.g., AAV) particle, wherein the viral particle transduces a nucleic acid molecule (e.g., an scAAV) encoding two or more (e.g., two, three, four, five, or six) gRNAs into a population of HSPCs (e.g., HSCs and/or HPCs); and modifying the genome of the population of HSPCs (e.g., HSCs and/or HPCs) with a sequence-targeting nuclease. The type of virus is not limited and may be any described herein. Each gRNA may be capable of directing a sequence-targeting nuclease (which may be delivered separately, as described herein) to a target locus in the genome of an HSPC (e.g., HSC and/or HPC). The method may further include administering to the subject a second viral (e.g., AAV) particle, wherein the viral particle transduces a nucleic acid molecule (e.g., an scAAV) encoding the sequence-targeting nuclease in a population of HSPCs (e.g., HSCs and/or HPCs). When the viral particle transduces the nucleic acid molecule into the HSPC (e.g., HSC and/or HPC), the encoded sequence-targeting nuclease and two or more gRNAs are expressed. Once expressed, the sequence-targeting nuclease and a gRNA form a complex capable of modifying a target locus in the genome of the HSPC (e.g., HSC and/or HPC).

In another embodiment, the methods provided herein include the steps of administering to the subject a viral (e.g., AAV) particle, wherein the viral particle transduces a nucleic acid molecule (e.g., an scAAV) encoding a sequence-targeting nuclease and two or more (e.g., two, three, four, five, or six) gRNAs into a population of HSPCs (e.g., HSCs and/or HPCs); and modifying the genome of the population of HSPCs (e.g., HSCs and/or HPCs) with the sequence-targeting nuclease. The type of virus is not limited and may be any described herein. When the viral particle transduces the nucleic acid molecule into the HSPC (e.g., HSC and/or HPC), the encoded sequence-targeting nuclease and two or more gRNAs are expressed. Once expressed, the sequence-targeting nuclease and a gRNA form a complex capable of modifying a target locus in the genome of the HSPC (e.g., HSC and/or HPC).

Steps of administering a nucleic acid molecule encoding the sequence-targeting nuclease may be substituted with the administering of a sequence-targeting nuclease polypeptide instead. Such polypeptide may be packaged and delivered in a liposome or lipid nanoparticle (LNP).

Any of the nucleic acid molecules described herein (e.g., an mRNA, plasmid, or viral vector) and/or sequence-targeting nuclease polypeptides described herein may be packaged into a delivery vehicle suitable for intravascular administration, such as a liposome or LNP. Liposomes are artificially-prepared vesicles composed of a lipid bilayer. The liposome may be, for example, a multilamellar vesicle (MLV) which may be hundreds of nanometers in diameter and may contain a series of concentric bilayers separated by narrow aqueous compartments; a small unicellular vesicle (SUV) which may be smaller than 50 nm in diameter; and a large unilamellar vesicle (LUV) which may be between 50 and 500 nm in diameter. Liposomes that have been previously described and shown to be suitable for delivery of a nucleic acid molecule (e.g., in vitro and in vivo) include those describe in, e.g., Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; all of which are incorporated herein in their entireties. LNPs are spherical vesicles made of ionizable lipids, which are positively charged at low pH and neutral at physiological pH. LNPs may have reduced toxicity as compared to liposomes.

Delivery of the nucleic acid molecule (e.g., an mRNA, plasmid, or viral vector, e.g., an AAV, e.g., an scAAV) and/or sequence-targeting nuclease polypeptides to the HSPCs (e.g., HSCs and/or HPCs) of the subject may be done in vivo or in situ with any intravascular administration procedure, such as by an intravenous injection or infusion. The method of treatment may treat, ameliorate, or prevent a disease or disorder, such as a hemoglobinopathy (e.g., SCD or β-thalassemia), a primary immunodeficiency (e.g., X-SCID, ADA-SCID, WAS, or CGD), a viral infection (e.g., a HIV, HHV, or CMV infection), a cytopenia (e.g., FA or SDS), or a storage or metabolic disorder (e.g., Gaucher disease or X-ALD).

Combinations of nucleic acid molecules may be employed. For example, gRNAs may be delivered via a viral vector, e.g., scAAV, and the sequence-targeting nuclease may be delivered via a different viral vector, mRNA, or plasmid. When two different nucleic acids are employed, they may be administered together or in sequence. When administered together, the two different nucleic acids may be packaged together in a larger structure, such as liposome or LNP.

### Nucleic Acid Molecules

The methods described herein utilize a nucleic acid molecule for the expression of a plurality of gRNAs (e.g., two to six gRNAs, e.g., two, three, four, five, or six gRNAs) and/or the expression of a plurality of sequence-targeting nucleases. In some embodiments, each gRNA sequence is present at least twice in the nucleic acid molecule. For example, each gRNA sequence may be present 2, 3, 4, 5, or 6 times in the nucleic acid molecule. The nucleic acid molecule may be a DNA molecule, RNA molecule, mRNA molecule, plasmid, or a viral vector.

Suitable viruses for use in generating the viral vectors described herein include, e.g., adenoviruses, AAVs (e.g., scAAVs), retroviruses (e.g., lentiviruses), vaccinia virus and other poxviruses, herpesviruses (e.g., herpes simplex virus), and others. The virus may or may not contain sufficient viral genetic information for production of infectious virus when introduced into host cells, i.e., viral vectors may be replication-competent or replication-defective.

In some embodiments, the virus is AAV serotype 1, 2, 6, 8, 9, 10 or Anc80 (i.e., Anc80L65) (disclosed in WO2015054653, incorporated herein by reference). Any AAV serotype, or modified AAV serotype, may be used as appropriate and is not limited.

Another suitable AAV may be, e.g., rh10 ([WO 2003/042397). Still other AAV sources may include, e.g., AAV9 (U.S. Pat. No. 7,906,111; US 2011-0236353-A1), and/or hu37 (see, e.g., U.S. Pat. No. 7,906,111; US 2011-0236353-A1), AAV1, AAV2, AAV3, AAV4, AAVS, AAV6, AAV6.2, AAV7, AAV8, (U.S. Pat. Nos. 7,790,449; 7,282,199) and others. See, e.g., WO 2003/042397; WO 2005/033321, WO 2006/110689; U.S. Pat. Nos. 7,790,449; 7,282,199; 7,588,772B2 for sequences of these and other suitable AAV, as well as for methods for generating AAV vectors. Still other AAV may be selected, optionally taking into consideration tissue preferences of the selected AAV capsid. A recombinant AAV vector (AAV viral particle) may comprise, packaged within an AAV capsid, a nucleic acid molecule containing a 5′ AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ ITR. Alternatively, the AAV vector may contain a shortened AAV 5′ inverted terminal repeat (ITR) and/or a shortened 3′ ITR in which the D-sequence and/or terminal resolution site (trs) of the ITR(s) are deleted.

A single-stranded AAV viral vector may be used in the methods described here. Methods for generating and isolating AAV viral vectors suitable for delivery to a subject are known in the art (e.g., See U.S. Pat. Nos. 7,790,449; 7,282,199; WO 2003/042397; WO 2005/033321, WO 2006/110689; and U.S. Pat. No. 7,588,772 B2).

In particular, the AAV vector used in the methods described herein may be a scAAV. Self-complementary AAV refers to a construct in which a coding region carried by a recombinant AAV nucleic acid sequence has been designed to form an intra-molecular double-stranded DNA template. Upon infection, rather than waiting for cell mediated synthesis of the second strand, the two complementary halves of the scAAV will associate to form one double stranded DNA (dsDNA) unit that is ready for immediate replication and transcription (e.g., see D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254). Self-complementary AAVs are described in, e.g., U.S. Pat. Nos. 6,596,535; 7,125,717; and 7,456,683, each of which is incorporated herein by reference in its entirety.

The scAAV may be about 1 kilobase (kb) to about 3.3 kb in length (e.g., about 1 kb to about 2.5 kb, about 1.5 kb to about 3 kb, about 2 kb to about 2.5 kb, about 2 kb to about 2.3kb, about 2.5 kb to about 3 kb or about 2.7 kb to 3.3 kb). For example, the scAAV may be about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, or about 3.3 kb.

The viral vector can also have certain modification such as 2′-deoxy, 2′-deoxy-2′fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

The viral vector (e.g., scAAV) may contain one or more promoters capable of directing expression of a transgene (e.g., expression of a sequence-targeting nuclease and/or one or more gRNAs). While any promoter is envisioned exemplary promoters include a cytomegalovirus (CMV) promoter, retrovirus promoter, simian virus (e.g., SV40) promoter, papilloma virus promoter, herpes virus promoter, (or other virus that infects mammalian cells), human elongation factor-1 alpha (EF1α), ubiquitin promoter (e.g., ubiquitin B or C), globin promoter, actin globin promoter, phosphoglycerate kinase (PGK) globin promoter, or CAG promoter. A CAG promoter combination of the CMV early enhancer element and chicken beta-actin promoter. In some embodiments a human promoter may be used. In some embodiments, the promoter is selected from a CMV promoter, U6 promoter, EF1α, an H1 promoter, a constitutive promoter, and a ubiquitous promoter. In some embodiments, the promoter directs expression in a particular cell type (e.g., an HSC specific promoter). The same promoter may be operably linked to all gRNAs present in the viral vector (e.g., the scAAV), or each gRNA of the vector may be operably linked to a distinct promotor. For example, the nucleotide sequence of the viral vector (e.g., the scAAV) may include one, two, three, four, five, or six distinct promoters that controls, two, three, four, five, or six separate gRNAs.

The viral vector (e.g., AAV, e.g., scAAV) may encode a sequence-targeting nuclease and/or two or more, e.g., two to six gRNAs (e.g., two to three gRNAs, two to four gRNAs, two to five gRNAs, or two to six gRNAs, e.g., two gRNAs, three gRNAs, four gRNAs, five gRNAs, or six gRNAs), as described further below.

### Sequence-Targeting Nucleases

The sequence-targeting nucleases that can be used in the methods disclosed herein are not limited and may be any sequence-targeting nuclease known in the art. For example, the sequence-targeting nuclease may be a Zinc-Finger Nuclease (ZFN), a Transcription activator-like effector nuclease (TALEN), an RNA-guided nuclease (e.g., a Cas nuclease (e.g., Cas9 nuclease)), or a functional fragment or functional variant thereof.

ZFNs and TALENs contain the nuclease domain of the restriction enzyme FokI (or an engineered variant thereof) fused to a site-specific DNA binding domain (DBD) that is appropriately designed to target the nuclease to a target locus. In the case of ZFNs, the DNA binding domain (DBD) contains a zinc finger DBD. In the case of TALENs, the site-specific DBD is designed based on the DNA recognition code employed by transcription activator-like effectors (TALEs), a family of site-specific DNA binding proteins found in plant-pathogenic bacteria such as Xanthomonas species.

The Clustered Regularly Interspaced Short Palindromic Repeats (CRISPR) system (e.g., of Type II or of Type IV) is a bacterial adaptive immune system that has been modified for use as an RNA-guided endonuclease technology for genome engineering. The bacterial system comprises two endogenous bacterial RNAs called crRNA and tracrRNA and a CRISPR-associated (Cas) nuclease (e.g., Cas9 or Cas12a). The tracrRNA has partial complementarity to the crRNA and forms a complex with it. The Cas protein is guided to the target sequence by the crRNA/tracrRNA complex, which forms a RNA/DNA hybrid between the crRNA sequence and the complementary sequence in the target. For use in genome modification, the crRNA and tracrRNA components are often combined into a single chimeric guide RNA (sgRNA or gRNA) in which the targeting specificity of the crRNA and the properties of the tracrRNA are combined into a single transcript that localizes the Cas protein to the target sequence so that the Cas protein can cleave the DNA. The gRNA often comprises about a 20 nucleotide (nt) (e.g., 18 nt, 19 nt, 20 nt, 21 nt, or 22 nt) guide sequence complementary or homologous to the desired target sequence followed by about 80 nt (e.g., 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, 85 nt, 86 nt, 87 nt, 88 nt, 89 nt, 90 nt, 91 nt, 92 nt, 93 nt, 94 nt, 95 nt, or 96 nt) of hybrid crRNA/tracrRNA. The Cas protein will be directed to any DNA sequence (e.g., target locus) with the correct target sequence and, in some cases, a PAM sequence. The PAM sequence varies depending on the species of bacteria from which the Cas protein was derived.

Exemplary Cas nuclease that can be used in the methods described herein include Cas1, Cas2, Cas3, Cas4, Cas5, Cas6, Cas7, Cas8, Cas9 and Cas10, Cas12a, (e.g., Cpf1), Cas12b (e.g., C2c1), Cas12c (e.g., C2c3), Cas12d (e.g., CasY), Cas12e (e.g., Cas12X), Cas12f (e.g., Cas14), Cas12j (e.g., Caso), Csn2, SauriCas9, CasMINI (e.g., see Xu et al., Molecular Cell, 81(20): 4333-4345 (2021)), or AsCas12f1 (e.g., see Wu et al., Nature, 17:1132-1138 (2021)). In some embodiments, the site specific nuclease is a Cas9 protein. For example, Cas9 from Streptococcus pyogenes (Sp), Neisseria meningitides (Nme), Staphylococcus aureus (Sa), Streptococcus thermophiles (St), Staphylococcus auriculari s(Sauri), or Treponema denticola (Td) may be used. The PAM sequences for these Cas9 proteins are (from 5′ to 3′) NGG, NNNNGATT, NNAGAA, NNRGAA (wherein R is A or G), NNGG, and NAAAAC, respectively. In some embodiments, the Cas9 is from Staphylococcus aureus (e.g., saCas9). In some embodiments, the Cas9 is from Neisseria meningitides (NmeCas9). In some embodiments, the Cas9 is from Streptococcus thermophiles (StCas9). In some embodiments, the Cas9 is from Staphylococcus auricularis (SauriCas9). In some embodiments, the Cas9 is from Treponema denticola (TdCas9). In some embodiments, the Cas9 is from Campylobacter jejuni. In some embodiments, the Cas12f1 is from Ruminiclostridium herbifermentans. In some embodiments, the Cas12f is from Oscillibacter sp.

A number of engineered variants of the site-specific nucleases have been developed and may be used in the methods herein. For example, engineered variants of Cas9 and FokI are known in the art. Furthermore, it will be understood that a biologically active fragment or variant nuclease can be used. Other variations include the use of hybrid site specific nucleases. For example, in CRISPR RNA-guided Fold nucleases (RFNs) the FokI nuclease domain is fused to the amino-terminal end of a catalytically inactive Cas9 protein (dCas9) protein. RFNs act as dimers and utilize two guide RNAs (Tsai, Q S, et al., Nat Biotechnol. 2014; 32(6): 569-576). Site-specific nucleases that produce a single-stranded DNA break are also of use for genome editing. Such nucleases, sometimes termed “nickases” can be generated by introducing a mutation (e.g., an alanine substitution) at key catalytic residues in one of the two nuclease domains of a site specific nuclease that comprises two nuclease domains (such as ZFNs, TALENs, and Cas proteins). Examples of such mutations include D10A, N863A, and H840A in SpCas9 or at homologous positions in other Cas9 proteins. A nick can stimulate HDR at low efficiency in some cell types. Two nickases, targeted to a pair of sequences that are near each other and on opposite strands can create a single-stranded break on each strand (“double nicking”), effectively generating a DSB. In some embodiments, the Cas protein is a SpCas9 variant. In some embodiments, the SpCas9 variant is a R661A/Q695A/Q926A triple variant or a N497A/R661A/Q695A/Q926A quadruple variant (e.g., see Kleinstiver et al., Nature, Vol. 529, pp. 490-495 (and supplementary materials) (2016), which is incorporated herein by reference in its entirety). In some embodiments, the Cas protein is C2c1, a class 2 type V-B CRISPR-Cas protein (e.g., see Yang et al., Cell, Vol. 167, pp. 1814-1828 (2016), which is incorporated herein by reference in its entirety). In some embodiments, the Cas protein is Cpf1 (also known as Cas12a), a class 2 type V-A CRISPR-Cas protein. In some embodiments, the Cas protein is one described in US 20160319260, which is incorporated herein by reference.

In some embodiments, the sequence-targeting nuclease has at least about 80%, 85%, 90%, 91%, 92%, 93%, 94%, 95%, 96%, 97%, 98% or 99% polypeptide sequence identity to a naturally occurring sequence-targeting nuclease. The nucleic acid encoding the sequence-targeting nuclease should be sufficiently short to be included in the virus (e.g., AAV). The sequence targeting nuclease may be engineered to be a truncated or miniaturized nuclease, such as the miniaturized Cas12f nuclease, CasMINI (e.g., see Xu et al., Molecular Cell, 81(20): 4333-4345 (2021), which is incorporated herein by reference), the AsCas12f1 nuclease described in Wu et al., Nature, 17:1132-1138 (2021), or any others known in the art.

When encoded by a viral vector described herein the sequence-targeting nuclease may be about 400 amino acids (aa) to about 2000 aa in length (e.g., about 400 aa to about 1000 aa, about 500 aa to about 1000 aa, about 550 aa to about 1120 aa, about 750 aa to about 1500 aa, or about 1000 aa to about 2000 aa in length). When encoded by a viral vector described herein the sequence-targeting nuclease may be about 1000 aa to about 1500 aa in length (e.g., about 1000 aa to about 1100 aa, about 1000 aa to about 1200 aa, about 1000 aa to about 1300 aa, about 1000 aa to about 1400 aa, or about 1000 aa to about 1500 aa in length). When encoded by a viral vector described herein the sequence-targeting nuclease may be about 500 aa to about 1000 aa in length (e.g., about 400 aa to about 500 aa, about 500 aa to about 600 aa, about 500 aa to about 700 aa, about 500 aa to about 800 aa, about 500 aa to about 900 aa, or about 500 aa to about 1000 aa in length).

### Guide RNAs (gRNAs)

The methods described herein utilize a viral vector (e.g., an AAV) that encodes two or more gRNAs. Any number of gRNAs capable of being expressed by a viral vector is envisioned. For example, the viral vector may be an AAV (e.g., a scAAV) that encodes two to three gRNAs, two to four gRNAs, two to five gRNAs, or two to six gRNAs (e.g., two gRNAs, three gRNAs, four gRNAs, five gRNAs, or six gRNAs). Each gRNA may be under the control of a promoter. While any promoter is envisioned, exemplary promoters include a CMV promoter, retrovirus promoter, simian virus (e.g., SV40) promoter, papilloma virus promoter, herpes virus promoter, (or other virus that infects mammalian cells), EF1α promoter, ubiquitin promoter (e.g., ubiquitin B or C), globin promoter, actin globin promoter, PGK globin promoter, or CAG promoter. In some embodiments a human promoter may be used. In some embodiments, the promoter is selected from a CMV promoter, U6 promoter, EF1α, an H1 promoter, a constitutive promoter, and a ubiquitous promoter. In some embodiments, the promoter directs expression in a particular cell type (e.g., an HSC specific promoter).

Each gRNA being expressed may be under the control of a unique promoter sequence. By way of example, a scAAV may encode a first gRNA operably linked to a CAG promoter, a second gRNA operably linked to a CMV promoter, and a third gRNA operably linked to an EF1 α promoter. In another example, a scAAV may encode a first gRNA operably linked to a U6 promoter, a second gRNA operably linked to an H1 promoter, and a third gRNA operably linked to an 7SK promoter. Alternatively, each gRNA being expressed may be under the control of the same type of promoter. By way of example a scAAV may encode a first gRNA operably linked to a first CAG promoter and a second gRNA operably linked to a second CAG promoter.

One of ordinary skill in the art appreciates that the gRNA need not be perfectly complementary or homologous to the target sequence. For example, in some embodiments it may have one or two mismatches. The genomic sequence which the gRNA hybridizes is typically flanked on one side by a Protospacer Adjacent Motif (PAM) sequence although one of ordinary skill in the art appreciates that certain Cas proteins may have a relaxed requirement for a PAM sequence. The PAM sequence is present in the genomic DNA but not in the gRNA sequence. The Cas protein may be directed to any DNA sequence with the correct target sequence and PAM sequence. The PAM sequence varies depending on the species of bacteria from which the Cas protein was derived.

A gRNA is generally about 20 nt to about 300 nt in length (e.g., about 20 nt to about 50 nt, about 25 nt to about 75 nt, about 30 nt to about 100 nt, about 50 nt to about 150 nt, about 75 nt to about 175 nt, or about 100 nt to about 200 nt in length). A gRNA may contain a spacer sequence containing a plurality of bases complementary to a protospacer sequence in the target locus. The spacer sequence of a gRNA enables sequence-specific targeting of a sequence-targeting nuclease to its target locus by hybridizing to the target locus. The spacer sequence may be 50%, 60%, 75%, 80%, 85%, 90%, 95%, 97.5%, 98%, 99%, or more complementary to its intended target locus.

For introducing an indel into a target locus, at least two gRNAs (e.g., two to three gRNAs, two to four gRNAs, two to five gRNAs, or two to six gRNAs, e.g., two gRNAs, three gRNAs, four gRNAs, five gRNAs, or six gRNAs) having a first spacer sequence maybe be used. Additional gRNAs having a second, third, or fourth, spacer sequence are envisioned.

For excising a target locus from the HSPC (e.g., HSC and/or HPC) genome, at least two gRNAs (e.g., two to three gRNAs or two to four gRNAs, e.g., two gRNAs, three gRNAs, or four gRNAs) having a first spacer sequence and at least two gRNAs (e.g., two to three gRNAs or two to four gRNAs, e.g., two gRNAs, three gRNAs, or four gRNAs) having a second spacer sequence may be used. Additional gRNAs having a third, fourth, etc. spacer sequence are envisioned as well. The first spacer sequence may have 50%, 60%, 75%, 80%, 85%, 90%, 95%, 97.5%, 98%, 99%, or 100% complementarity to the 5′ end of the target locus. The second spacer sequence may have 50%, 60%, 75%, 80%, 85%, 90%, 95%, 97.5%, 98%, 99%, or 100% complementarity to the 3′ end of the target locus. If a third or fourth spacer sequence is utilized, it may be designed to have 50%, 60%, 75%, 80%, 85%, 90%, 95%, 97.5%, 98%, 99%, or 100% complementarity to any region within 5′ end and 3′ end of the target locus.

### Subjects

The subject may be a mammal, e.g., a primate, e.g., a human. In particular, the mammal can be a human, non-human primate, mouse, rat, dog, cat, horse, or cow, but are not limited to these examples. A subject can be male or female. A human subject may be between 1 day and 6 months old. A human subject may be between 6 months and 24 months old. A human subject may be between 2 years and 6 years, 6 years and 12 years, or 12 years and 18 years old. A human subject may be between 18 years and 30 years, 30 years and 50 years, 50 years and 80 years, or greater than 80 years old. For example, the subject is at least about 5, 10, 20, 30, 40, 50, 60, 65, 70, 75, 80, 85, or 90 years of age. In some embodiments, a subject is an adult. For purposes hereof, a human at least 18 years of age is considered an adult (e.g., greater than 18 years of age for a human subject). In some embodiments, the subject is a juvenile (e.g., less than 18 years of age for a human subject). In some embodiments a subject is an embryo. In some embodiments the subject is a fetus. In certain embodiments the subject is a pregnant female, wherein the female is administered the treatment in order to treat an embryo or fetus in utero.

The subject in need of treatment may have a genotype associated with a genetic blood disorder. The subject in need of treatment may be diagnosed with or exhibit symptoms of a genetic blood disorder. The subject in need of treatment may be one at risk of displaying symptoms of a genetic blood disorder because the subject has a genotype associated with a genetic blood disorder.

### Target Locus

The region of the HSPC (e.g., HSC and/or HPC) genome to be modified (e.g., target locus) may be in an intron, in an exon, or in a regulatory DNA element, such as a promoter, an enhancer region, a silencer region (i.e., a suppressor or repressor region), or an insulator region of the genome. The target locus may be in one allele or in both alleles of the subject. For example, the target locus may be the binding site of B-cell lymphoma/leukemia 11 (BCL11A), a repressor of fetal hemoglobin (HbF). In another example, to upregulate fetal hemoglobin in sickle cell disease, zinc finger protein 410 (ZNF410) can be disrupted using the methods described herein.

Modification of the target locus may treat, ameliorate, or prevent a disease in the subject by reducing gene expression of a target gene of interest or increasing gene expression of a target gene of interest, e.g., by disruption of a silencer or suppressor element.

Modification of the target locus (e.g., a suppressor element) may increase expression of a gene of interest by 5%, 10%, 15, 20%, 25%, 30%, 35%, 40%, 45%, 50%, 55%, 60%, 65%, 70%, 75%, 80%, 85%, 90%, 95%, 100%, or more (e.g., 125%, 150%, 175%, 200%, 250%, 300%, or more). Alternatively, modification of the target locus (e.g., a gene, a promoter, or an enhancer element) may decrease expression of a target gene by 5%, 10%, 15, 20%, 25%, 30%, 35%, 40%, 45%, 50%, 55%, 60%, 65%, 70%, 75%, 80%, 85%, 90%, 95%, or 100%. For example, disruption of a suppressor element for HbF increases the expression of HbF, which may combine with HbA to produce functional hemoglobin, e.g., in subjects with SCD or β-thalassemia, thereby treating the disease.

Using scAAV described herein with the methods described herein, at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 5-fold, 10-fold, 20-fold, 50-fold, 100-fold, or more of the HSPCs (e.g., HSCs and/or HPCs) are modified as compared to ssAAV delivery methods.

The target locus, or genotype therein, may be associated or correlated with a disease or disorder, such as a blood disorder. In some embodiments, the blood disorder is a genetic blood disorder. In some embodiments, the blood disorder is a monogenic blood disorder. In some embodiments, the blood disorder is a multigenic blood disorder. In some embodiments, the blood disorder is a disorder associated with one or more SNPs.

### Pharmaceutical Compositions

Any of the nucleic acid molecules (e.g., an mRNA, plasmid, or viral vector) describe herein, either naked or packaged (e.g., packaged into a viral particle, liposome, or LNP) may be formulated as a pharmaceutical composition for intravascular injection (e.g., intravenous injection or intravenous infusion). Pharmaceutical formulations may include any pharmaceutically acceptable carrier, excipient, or diluent. Exemplary excipients include, but are not limited to, any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, and/or combinations thereof. Remington's The Science and Practice of Pharmacy, 21st Edition, A. R. Gennaro (Lippincott, Williams & Wilkins, Baltimore, MD, 2006; incorporated herein by reference) discloses various pharmaceutical formulations and known techniques for the preparation thereof.

Pharmaceutical compositions may be provided as a kit for treating a blood disease in a subject described herein. The kit may contain two vials or other containers for the components. The kit includes a nucleic acid molecule described herein (e.g., an scAAV) that encodes at least two (e.g., two, three, four, five, or six) gRNAs, optionally in a pharmaceutically acceptable carrier, excipient, or diluent. The gRNAs may be capable to directing a sequence-targeting nuclease to a target locus in the genome of an HSC, and HPC, or a population of HSPCs. The kit also include a nucleic acid molecule described herein (e.g., an scAAV) that encodes a sequence-targeting nuclease described herein or a polypeptide of a sequence-targeting nuclease, optionally in a pharmaceutically acceptable carrier, excipient, or diluent. Optionally, the polypeptide of the sequence targeting nuclease may be packaged into a liposome or LNP molecule. The components of the kit may be formulated for intravenous administration (e.g., intravenous infusion).

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

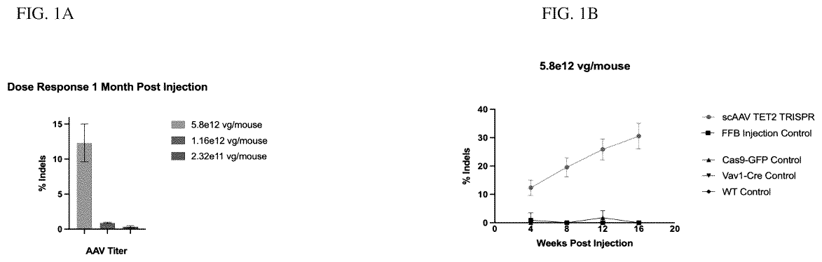

As shown in FIGS. 1A and 1B, scAAVs transduce HSCs in vivo and can enable CRISPR-induced gene edits. Administration in this example is to adult mice endogenously expressing SpCas9 in the blood lineage only (via the Vav1-iCre; LSL-Cas9-Gfp transgenic mouse system), and the target is Tet2.

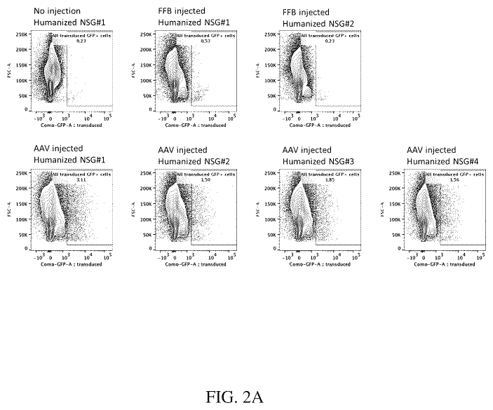

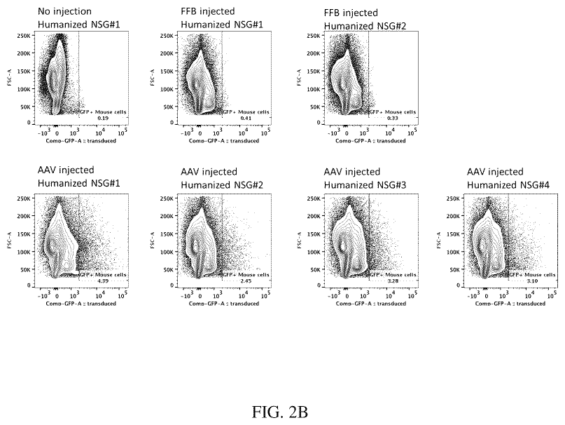

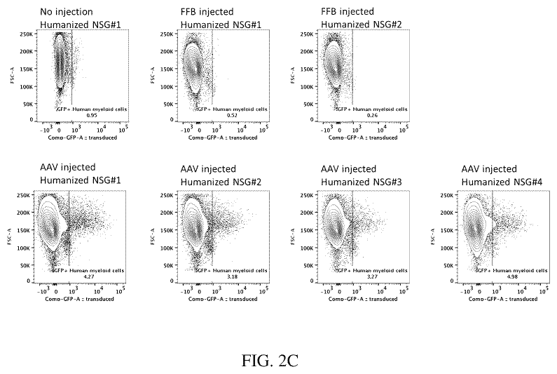

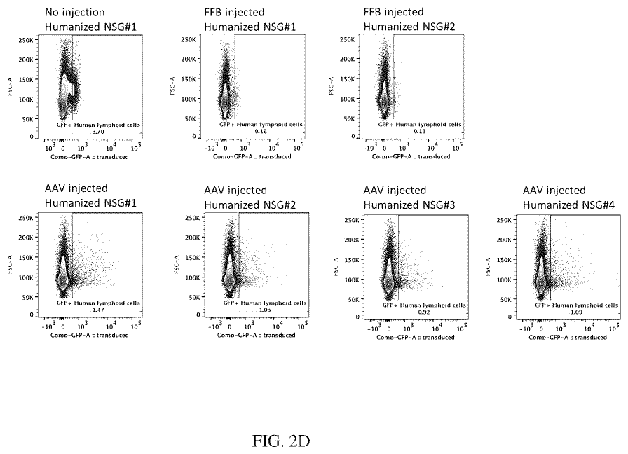

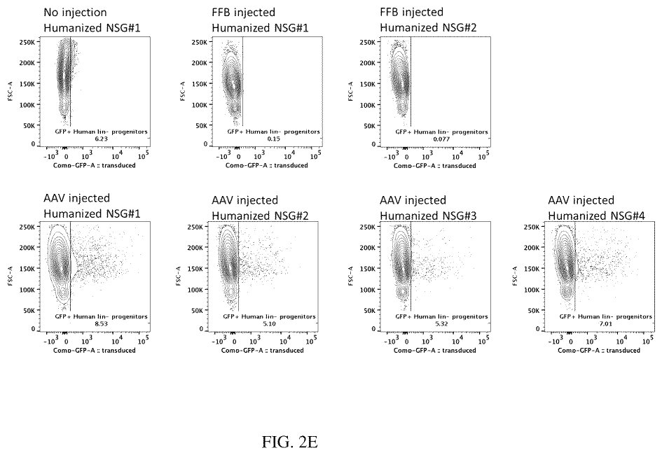

The data shown in FIG. 2 demonstrate transduction of both human and mouse bone marrow cells in adult humanized mice (mice transplanted with human CD34+ progenitors so that they have circulating human blood cells and HSCs) by systemically administered self-complementary AAV8 carrying a CBh promoter-GFP cargo (to mark transduced cells with green fluorescence). GFP+ bone marrow cells can be seen in the AAV-injected but not control (non-injected and FFB vehicle injected) humanized mice. GFP+ cells include human cells and mouse cells and mature lineages and lin-progenitors. FIG. 3 provides a summary of the data above, showing GFP transduction by scAAV8-Gfp in various human blood cell lineages (including progenitors that contain HSCs) and mouse blood cells.

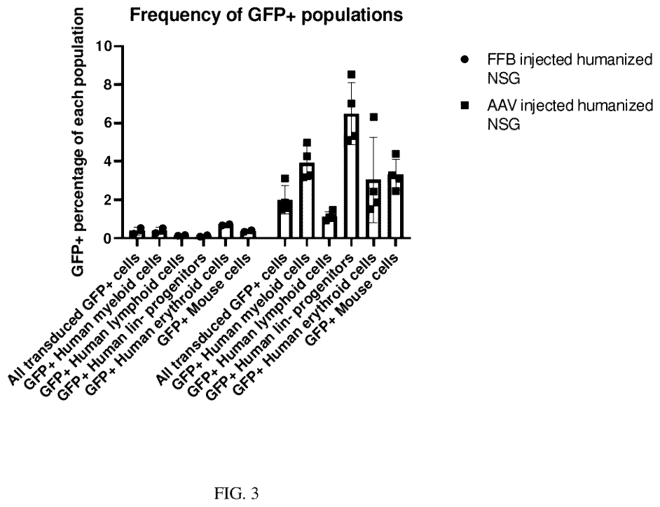

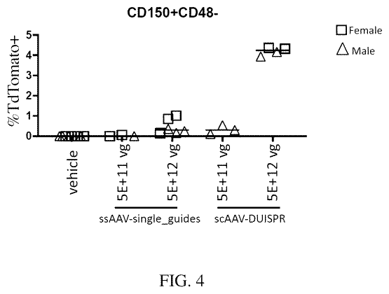

As shown in FIG. 4, neonatal Sp-Cas9-expressing Ai9 mice (harboring a Lox-STOP-Lox allele upstream of tdTomato and transgenic expression of a SpCas9-GFP cassette) were injected with the indicated dose of either single stranded AAV (ssAAV) encoding 1 copy of the gRNA targeting upstream and 1 of the gRNA targeting downstream of the STOP cassette (single) or with self-complementary AAV (scAAV) encoding 2 copies of the gRNA targeting upstream and 2 copies of the gRNA targeting downstream of the STOP cassette. One month later, CD150+CD48-hematopoietic stem cells were identified by flow cytometry and assessed for tdTomato expression (an indicator of successful CRISPR gene editing at the Ai9 locus). Editing rates were substantially higher with the scAAV+2x gRNA format and reached 4% of HSCs at the highest vg dose. Editing rates with the ssAAV+1x gRNA format and editing rates with lower doses of scAAV+2x gRNAs typically ranged from 0.2-1%. AAV serotype=AAV8.

### Example 1: In Vivo Gene Editing of Hematopoietic Stem Cells Via AAV-Delivered CRISPR Guide Rnas

Efficient in vivo gene editing of hematopoietic stem cells (HSCs) has the potential to eliminate the requirement for hematopoietic stem cell transplantation (HSCT) while leveraging the potency of HSCs for blood cell repopulation in treating monogenic blood disorders. Towards this aim, adeno-associated virus (AAV) was used to deliver CRISPR guide RNAs (gRNAs) to gene edit HSCs in vivo in two Cas9-expressing transgenic mouse models (e.g., an Ai9;Cas9-eGFP mouse model and an SCD;Cas9-eGFP mouse model). AAVs were chosen as a delivery vehicle because they have low immunogenicity compared to other viral vectors as well as non-integration characteristics, which eliminates the possibility of insertional oncogenesis.

### Experimental Methods

### AAV Cloning and Production

AAV backbones were cloned using Gibson cloning and commercially-available plasmids. AAV production for Ai9;Cas9 studies were outsourced to Penn Vector Core at University of Pennsylvania. AAV production for SCD;Cas9 studies was performed in-house.

### Animal Models

Ai9 and Cas9-eGFP animals are available for purchase from Jackson Laboratory. Ai9 animals expressing Cas9-eGFP are generated from this cross. Both Ai9 and Cas9 transgenes are in the Rosa26 locus. Townes animals (sickle cell disease model) were purchased from Jackson Laboratory. Cas9-expressing Townes animals (SCD;Cas9) were generated in-house through breeding and selecting for mice with desired loci. SCD;Cas9 animals exclusively express human globin genes (no mouse genes) as well as constitutive Cas9-eGFP expression from transgene in the Rosa26 locus.

### Results

### Adult Ai9;Cas9-eGFP Model

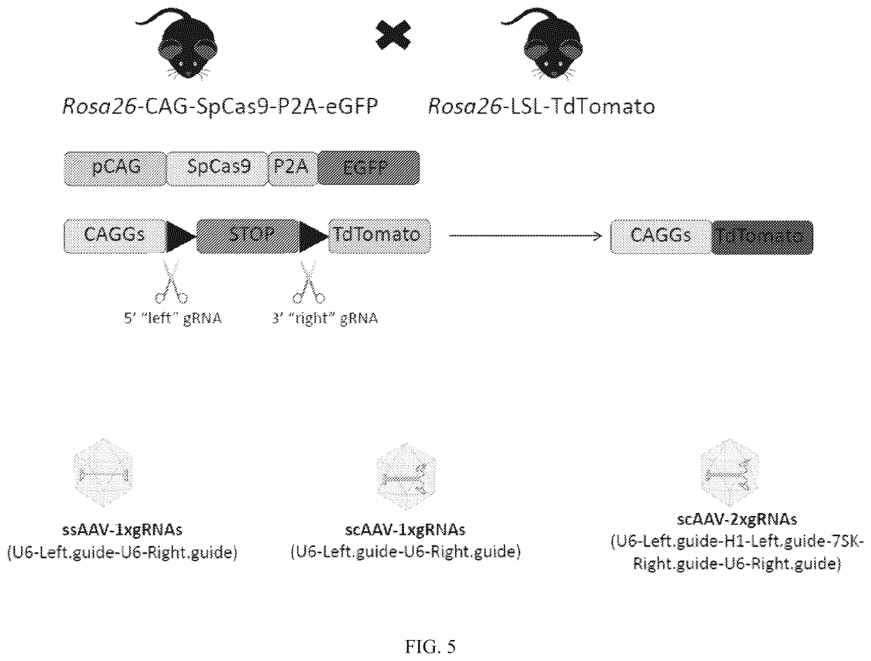

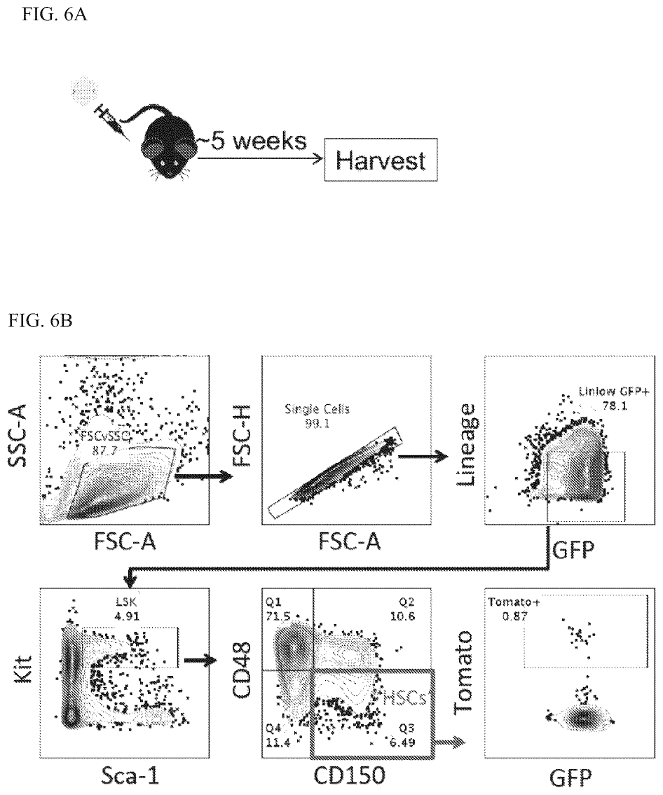

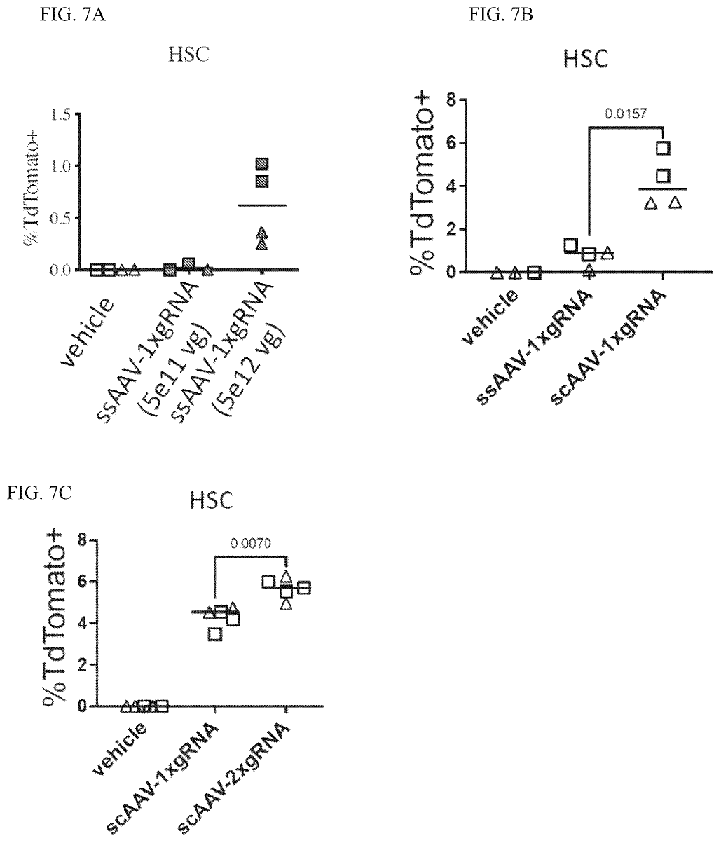

To assess gene editing in hematopoietic stem and progenitor cells (HSPCs) in vivo, we delivered AAVs encoding gRNAs to target removal of the Ai9 LSL into Ai9;SpCas9-eGFP mice (FIG. 5, top). This approach enabled us to test multiple different gRNA vector configurations and AAV formats in order to identify optimal conditions for in vivo CRISPR-mediated gene editing (FIG. 5, bottom). Our general approach for determining gene modification in HSCs consists of systemic AAV delivery followed by analysis of bone marrow (BM) HSCs for TdTomato expression about 5 weeks post AAV injection (FIG. 6A). Phenotypic HSCs are assayed for TdTomato expression using flow cytometric analysis (FIG. 6B). Rare, but detectable, gene modified HSCs were found when 5×10¹² viral genomes (vg) of single stranded (ss)-AAV-1xgRNA was delivered to adult Ai9;SpCas9 animals (FIG. 7A). Therefore, we used 5×10¹² vg/animal in future experiments. We then compared ss and self-complementary (sc) configurations of AAVs. Conventional, ssAAV vectors rely on the host's cellular machinery for complementary strand synthesis, a rate-limiting step in the expression of AAV-encoded material. Alternatively, self-complementary AAVs (scAAVs) bypass this step by self-annealing. To determine whether bypassing complementary strand synthesis might enhance the effectiveness of AAV-delivered gene editing complexes in HSPCs, we cloned our Ai9 targeting gRNAs into a scAAV vector (scAAV8-1xgRNA.Ai9.STOP.guides) and compared editing rates seen upon in vivo delivery of this vector to those seen with ssAAV formats. We found that the use of scAAV vectors significantly improved editing rates in adult HSPCs (˜6-fold greater induction of the percent of TdTomato-positive (% TdTomato+) HSPCs) in comparison to ssAAV vectors (FIG. 7B). Building on this observation, we produced scAAV vectors encoding an additional copy of each of the gRNAs, with the original 5′- and 3′-targeting gRNAs driven by the hU6 promoter and the additional 5′- and 3′-targeting gRNAs driven by the H1 and 7SK promoters, respectively. This new vector (called scAAV-2xgRNAs) allowed us to test whether additional guide copies can enhance editing rates due to higher expression of gRNAs. Direct comparison of single copy gRNA vectors versus double copy gRNA designs in adult animals showed a ˜25% improvement in HSPC editing (FIG. 7C). Therefore, in vivo gene editing is more efficient when multiple copies of the gRNA are provided.

### Newborn Ai9; Cas9-eGFP Model

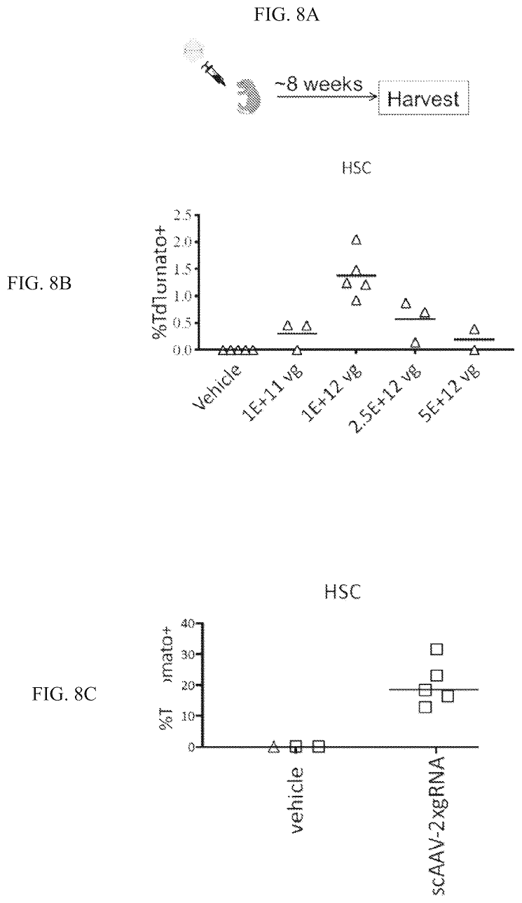

We also assessed levels of HSC gene modification in neonates upon delivery of our vectors (FIG. 8A). We found 1×10¹² vg/animal to be the most efficient dose for HSC gene modification using our ssAAV-1xgRNA vector (FIG. 8B). We found about 20% TdTomato+ HSPCs in neonates injected with scAAV-2xgRNAs, which is about a 15-fold improvement in HSC editing rate compared to our initial ssAAV-1xgRNAs vector (FIG. 8C). Therefore, our in vivo gene modification approach is even more efficient when injected into newborn animals.

### Adult SCD;Cas9-eGFP Model

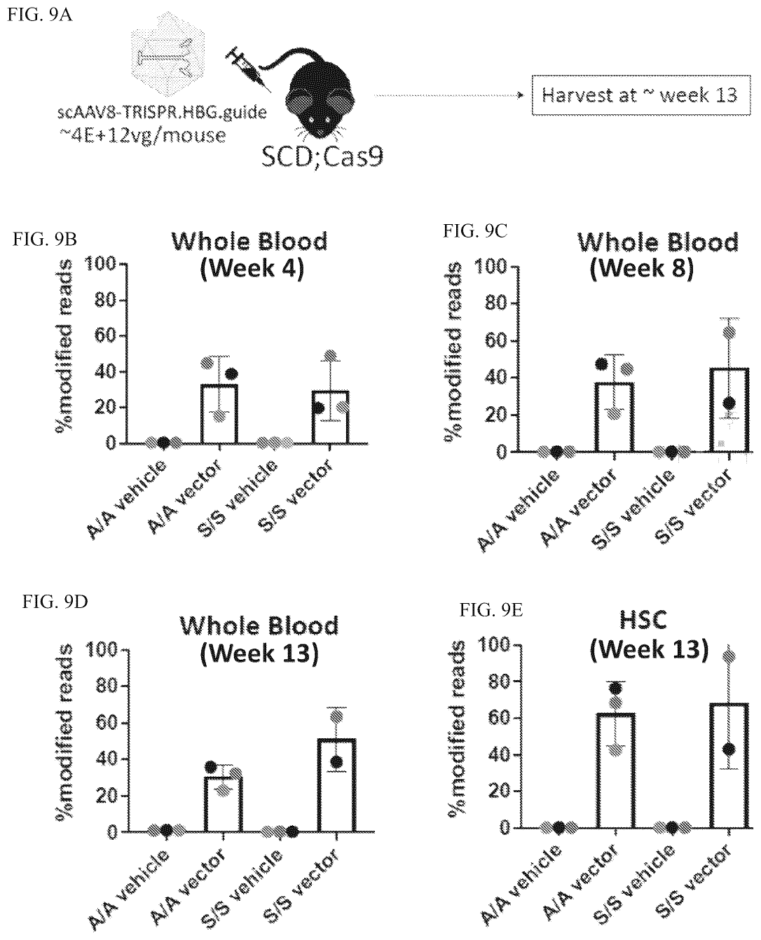

We then assessed our AAV delivery of CRISPR components for in vivo gene editing of HSPCs in the context of Sickle Cell Disease (SCD). For these studies, we made use of the “Townes” mouse model in which mouse alpha-and beta-globin genes are replaced with human hemoglobin-alpha, hemoglobin-gamma and sickle or non-sickle hemoglobin beta. We generated and utilized Townes animals with endogenous SpCas9-eGFP expression (Townes;SpCas9-eGFP). We verified that Townes;SpCas9-eGFP animals homozygous for sickle HBB (HBS) show SCD phenotypes comparable to the originally described Townes mice. Inducing fetal hemoglobin (HbF) is thought to ameliorate SCD complications. One approach for HbF induction is to disrupt the binding site of B-cell lymphoma/leukemia 11 (BCL11A; repressor of HbF) in the HBG promoter. HBG encodes gamma-globin, which forms fetal globin when assembled with hemoglobin alpha-chains. Therefore, we designed and produced scAAV-HBG.promoter.TRISPR.HBG.guide vectors; scAAV vector with 3 copies of gRNAs that target the BCL11A erythroid enhancer binding site. Each gRNA was driven by one of the U6, H1, or 7SK promoters. We injected scAAV-HBG.promoter.TRISPR.HBG.guide into adult Townes;SpCas9-eGFP animals carrying either two copies of HBS or HBB (sickle or non-sickle, respectively) (FIG. 9A). At 4 weeks (FIG. 9B), 8 weeks (FIG. 9C), and 13 weeks (FIG. 9D) post injection, gene modification was detected in the blood of mice. At 13 weeks post injection robust editing at the HBG promoter was observed in BM HSCs of vector-injected animals (both HBS and HBB homozygotes), whereas vehicle-injected animals show no editing (FIG. 9E). Therefore, we have successfully generated an in vivo HSC gene modification system capable of modifying a gene directly associated with human disease.

### Discussion

### Adaptability of System to Other Approaches

Here, we rely on induction of insertions/deletions (INDELs) in both of our mouse models (i.e. “cutting”). In the Ai9;Cas9 system, two cuts are required for excision of STOP cassette upstream of TdTomato. In the SCD;Cas9 model, disruption of binding site for a gene repressor (BCL11A) is utilized to upregulate HbF levels. Therefore, this system can be readily applied to any approach where disruption or excision of a target would be therapeutic.

### Size Limitation

A feature of our approach that makes it efficient is the use of scAAV vectors. The limiting factor for AAV vectors (in particular scAAV configuration) is cargo size, ˜4.7 kb for ssAAV and ˜2.3 kb for scAAV vectors. SaCas9 and SauriCas9 are variants that can be fit into the ssAAV configuration with proving efficiency in other systems. Miniature Cas versions (e.g. CasMINI and Cas12f1) have the potential to fit into scAAV vectors as well.

What is claimed is:

1. A method for treating a blood disorder in a subject comprising administering to the subject a nucleic acid molecule comprising a nucleotide sequence encoding two to six guide RNAs (gRNAs), wherein:
(a) the nucleic acid molecule is delivered to a population of hematopoietic stem and progenitor cells (HSPCs); and
(b) each gRNA is capable of directing a sequence-targeting nuclease to a target locus in the genome of the HSPCs.

2. A method for treating a blood disorder in a subject comprising administering to the subject a nucleic acid molecule comprising a nucleotide sequence encoding:
(a) two to six gRNAs; and
(b) a sequence-targeting nuclease,
wherein the nucleic acid molecule is delivered in a population of HSPCs and each gRNA is capable of directing the sequence-targeting nuclease to a target locus in the genome of the HSPC.

3. A method for removing a suppressor element in a subject comprising administering to the subject a nucleic acid molecule comprising a nucleotide sequence encoding two to six gRNAs, wherein:
(a) the nucleic acid molecule is delivered to a population of HSPCs; and
(b) each gRNA is capable of directing a sequence-targeting nuclease to a target locus in the genome of the HSPC.

4. A method for removing a suppressor element in a subject comprising administering to the subject a nucleic acid molecule comprising a nucleotide sequence encoding:
(a) two to six guide RNAs; and
(b) a sequence-targeting nuclease,
wherein the nucleic acid molecule is delivered to a population HSPC and each gRNA is capable of directing the sequence-targeting nuclease to a target locus in the genome of the HSPCs.

5. The method of any one of claims 1-4, wherein the HSPC is a hematopoietic stem cell (HSC) or a hematopoietic progenitor cell (HPC).

6. The method of any one of claims 1-5, wherein the nucleic acid molecule is an mRNA molecule, a plasmid, or a viral vector.

7. The method of claim 6, wherein the viral vector is an adeno-associated virus (AAV).

8. The method of claim 7, wherein the AAV is a self-complementary AAV (scAAV).

9. The method of claim 8, wherein the scAAV is about 1 kilobase (kb) to about 3.3 kb in length.

10. The method of claim 9, wherein the scAAV is about 1.8 kb to about 2.1 kb in length.

11. The method of any one of claims 1-10, wherein the nucleotide sequence encodes two gRNAs.

12. The method of any one of claims 1-10, wherein the nucleotide sequence encodes three gRNAs.

13. The method of any one of claims 1-10, wherein the nucleotide sequence encodes four gRNAs.

14. The method of any one of claims 1-10, wherein the nucleotide sequence encodes five gRNAs.

15. The method of any one of claims 1-10, wherein the nucleotide sequence encodes six gRNAs.

16. The method of any one of claims 11-15, wherein each of the gRNAs are operably linked to a different promoter.

17. The method of claim 16, wherein the promoter is a constitutive promoter.

18. The method of claim 16, wherein the promoter is a ubiquitous promoter.

19. The method of claim 17 or 18, wherein the promoter is a human promoter, a viral promoter, or a bacterial promoter.

20. The method of any one of claims 16-19, wherein the promoter is selected from the group consisting of: a cytomegalovirus (CMV) promoter, a retrovirus promoter, a simian virus promoter, a papilloma virus promoter, a herpes virus promoter, an elongation factor-1 alpha (EF1α) promoter, a ubiquitin promoter, a globin promoter, an actin globin promoter, a phosphoglycerate kinase (PGK) globin promoter, a CAG promoter, a U6 promoter, a 7SK promoter, and an H1 promoter.

21. The method of claim 20, wherein the promoter is selected from the group consisting of: the U6 promoter, the H1 promoter, and the 7SK promoter.

22. The method of claim 1 or 3, wherein the nucleic acid further comprises a nucleotide sequence encoding the sequence-targeting nuclease.

23. The method of claim 1 or 3, wherein the method further comprises administering to the subject a second nucleic acid molecule comprising a nucleotide sequence encoding the sequence-targeting nuclease.

24. The method of claim 1 or 3, wherein the method further comprises administering to the subject a polypeptide of the sequence-targeting nuclease, wherein the polypeptide in packaged into a liposome or lipid nanoparticle (LNP).

25. The method of any one of claims 1-24, wherein the sequence-targeting nuclease is a Clustered Regularly Interspaced Short Palindromic Repeats (CRISPR)-associated (Cas) nuclease.

26. The method of claim 25, wherein the Cas nuclease is about 400 amino acids to about 2000 amino acids in size.

27. The method of claim 26, wherein the Cas nuclease is about 550 amino acids to about 1120 amino acids in size.

28. The method of claim 26 or 27, wherein the Cas nuclease is selected from the group consisting of: a Cas9 nuclease, a Cas12a nuclease, and a Cas12f nuclease.

29. The method of claim 28, wherein the Cas9 nuclease is from Staphylococcus auricularis (Sa) or Streptococcus pyogenes (Sp).

30. The method of claim 28, wherein the Cas12a or Cas12f nuclease is from Lachnospiraceae bacterium (Lb).

31. The method of any one of claims 1-30, wherein the nucleic acid molecule is administered to the subject intravenously.

32. The method of claim 31, wherein the nucleic acid molecule is administered by intravenous infusion.

33. The method of any one of claims 1-32, wherein the blood disorder is selected from the group consisting of: a hemoglobinopathy, a primary immunodeficiency, a viral infection in the blood, a cytopenia, and a storage or metabolic disorder.

34. The method of claim 33, wherein:
(a) the hemoglobinopathy is sickle cell disease (SCD) or beta thalassemia (β-thalassemia);
(b) the primary immunodeficiency is X-linked severe combined immunodeficiency (X-SCID), adenosine deaminase severe combined immunodeficiency (ADA-SCID), Wiskott-Aldrich syndrome (WAS), or chronic granulomatous disease (CGD);
(c) the viral infection is a human immunodeficiency virus (HIV), human herpesvirus (HHV), or cytomegalovirus (CMV) infection in the blood;
(d) the cytopenia is Fanconi anemia (FA) or Shwachman-Diamond syndrome (SDS); or
(e) the storage or metabolic disorder is Gaucher disease or X-linked adrenoleukodystrophy (X-ALD).

35. The method of any one of claims 1-34, wherein after administration of the nucleic acid molecule, the target locus in the genome of the HSPC is disrupted by the nuclease activity of the sequence-targeting nuclease.

36. The method of claim 35, wherein the target locus is disrupted by an insertion or a deletion of a nucleotide in the target locus.

37. The method of claim 36, wherein the target locus is excised from the genome by nuclease activity at both the 5′ and 3′ end of the target locus.

38. The method of claim 36 or 37 wherein the target locus is an intron, an exon, or a regulatory DNA element.

39. The method of claim 38, wherein the DNA regulatory element is an enhancer region, a suppressor region, or an insulator region.

40. The method of claim 39, wherein the suppressor region is repressing expression of fetal hemoglobin (HbF) in the subject.

41. The method of claim 40, wherein the suppressor region is a binding site of B-cell lymphoma/leukemia 11 (BCL11A).

42. A composition comprising:
(a) a nucleic acid molecule comprising a nucleotide sequence encoding at least two gRNAs; and
(b) a pharmaceutically acceptable carrier, excipient, or diluent,
wherein each gRNA is capable of directing a sequence-targeting nuclease to a target locus in a genome of a population of HSPCs.

43. A composition comprising a nucleic acid molecule comprising a nucleotide sequence encoding:
(a) at least two gRNAs;
(b) a sequence-targeting nuclease; and
(c) a pharmaceutically acceptable carrier, excipient, or diluent,
wherein each gRNA is capable of directing the sequence-targeting nuclease to a target locus in a genome of a population of HSPCs.

44. A composition comprising:
(a) a nucleic acid molecule comprising a nucleotide sequence encoding at least two gRNAs;
(b) a sequence-targeting nuclease; and
(c) a pharmaceutically acceptable carrier, excipient, or diluent,
wherein each gRNA is capable of directing the sequence-targeting nuclease to a target locus in a genome of a population of HSPCs.

45. The composition of any one of claims 42-44, wherein the population of HSPC comprise an HSC and/or an HPC.

46. composition of any one of claims 42-45, wherein the nucleic acid molecule is an mRNA molecule, a plasmid, or a viral vector.

47. The composition of claim 46, wherein the viral vector is an adeno-associated virus (AAV)

48. The composition of claim 47, wherein the AAV is a self-complementary AAV (scAAV).

49. The composition of claim 48, wherein the scAAV is about 1 kilobase (kb) to about 3.3 kb in length.

50. The composition of claim 49, wherein the scAAV is about 1.8 kb to about 2.1 kb in length.

51. The composition of any one of claims 42-50, wherein the nucleotide sequence encodes two gRNAs.

52. The composition of any one of claims 42-50, wherein the nucleotide sequence encodes three gRNAs.

53. The composition of any one of claims 42-50, wherein the nucleotide sequence encodes four gRNAs.

54. The composition of any one of claims 42-50, wherein the nucleotide sequence encodes five gRNAs.

55. The composition of any one of claims 42-50, wherein the nucleotide sequence encodes six gRNAs.

56. The composition of any one of claims 51-55, wherein each of the gRNAs are operably linked to a different promoter.

57. The composition of claim 56, wherein the promoter is a constitutive promoter.

58. The composition of claim 56, wherein the promoter is a ubiquitous promoter.

59. The composition of claim 57 or 58, wherein the promoter is a human promoter, a viral promoter, or a bacterial promoter.

60. The composition of any one of claims 56-59, wherein the promoter is selected from the group consisting of: a CMV promoter, a retrovirus promoter, a simian virus promoter, a papilloma virus promoter, a herpes virus promoter, an EF1α promoter, a ubiquitin promoter, a globin promoter, an actin globin promoter, a PGK globin promoter, a CAG promoter, a U6 promoter, a 7SK promoter, and an H1 promoter.

61. The composition of claim 60, wherein the promoter is selected from the group consisting of: the U6 promoter, the H1 promoter, and the 7SK promoter.

62. The composition of claim 42 or 44, wherein the nucleic acid molecule further comprises a nucleotide sequence encoding the sequence-targeting nuclease.

63. The composition of claim 42 or 44, wherein the composition further comprises a second nucleic acid molecule having a nucleotide sequence encoding the sequence-targeting nuclease.

64. The composition of claim 42 or 44, wherein the sequence-targeting nuclease is packaged in a liposome or lipid nanoparticle (LNP) as a polypeptide.

65. The composition of any one of claims 42-64, wherein the sequence-targeting nuclease is a Cas nuclease.

66. The composition of claim 65, wherein the Cas nuclease is about 400 amino acids to about 2000 amino acids in size.

67. The composition of claim 66, wherein the Cas nuclease is about 550 amino acids to about 1120 amino acids in size.

68. The composition of claim 66 or 67, wherein the Cas nuclease is selected from the group consisting of: a Cas9 nuclease, a Cas 12a nuclease, and a Cas 12f nuclease.

69. The composition of claim 68, wherein the Cas9 nuclease is from Staphylococcus auricularis (Sa) or Streptococcus pyogenes (Sp).

70. The composition of claim 68, wherein the Cas12a or Cas12f nuclease is from Lachnospiraceae bacterium (Lb).

71. The composition of any one of claims 42-70, wherein the composition is formulated for intravenous administration to the subject.

72. The composition of claim 71, wherein the composition is formulated for intravenous infusion to the subject.

73. The composition of any one of claims 42-72, wherein the composition is for use in treating blood disorder is selected from the group consisting of: a hemoglobinopathy, a primary immunodeficiency, a viral infection in the blood, a cytopenia, and a storage or metabolic disorder.

74. The composition of claim 73, wherein:
(a) the hemoglobinopathy is SCD or β-thalassemia;
(b) the primary immunodeficiency is X-SCID, ADA-SCID, WAS, or CGD;
(c) the viral infection is an HIV, HHV, or CMV infection in the blood;
(d) the cytopenia is FA or SDS; or
(e) the storage or metabolic disorder is Gaucher disease or X-ALD.

75. A kit for treating a blood disorder in a subject comprising:
(a) a first nucleic acid molecule having a nucleotide sequence encoding at least two gRNAs, wherein each gRNA is capable of directing a sequence-targeting nuclease to a target locus in a genome of a population of HSPCs; and
(b) a sequence-targeting nuclease or second nucleic acid molecule encoding the sequence-targeting nuclease.

76. The kit of claim 75, wherein the HSPC is an HSC or an HPC.

77. The kit of claim 75 or 76 wherein the first or second nucleic acid molecule is an mRNA molecule, a plasmid, or a viral vector.

78. The kit of claim 77, wherein the viral vector is an AAV.

79. The kit of claim 78, wherein the AAV is an scAAV.

80. The kit of claim 79, wherein the scAAV is about 1 kb to about 3.3 kb in length.

81. The kit of claim 80, wherein the scAAV is about 1.8 kb to about 2.1 kb in length.

82. The kit of any one of claims 75-81, wherein the nucleotide sequence encodes two gRNAs.

83. The kit of any one of claims 75-81, wherein the nucleotide sequence encodes three gRNAs.

84. The kit of any one of claims 75-81, wherein the nucleotide sequence encodes four gRNAs.

85. The kit of any one of claims 75-81, wherein the nucleotide sequence encodes five gRNAs.

86. The kit of any one of claims 75-81, wherein the nucleotide sequence encodes six gRNAs.

87. The kit of any one of claims 82-86, wherein each of the gRNAs are operably linked to a different promoter.

88. The kit of claim 87, wherein the promoter is a constitutive promoter.

89. The kit of claim 87, wherein the promoter is a ubiquitous promoter. 90 The kit of claim 88 or 89, wherein the promoter is a human promoter, a viral promoter, or a bacterial promoter.

91. The kit of any one of claims 87-90, wherein the promoter is selected from the group consisting of: a cytomegalovirus (CMV) promoter, a retrovirus promoter, a simian virus promoter, a papilloma virus promoter, a herpes virus promoter, an elongation factor-1 alpha (EF1α) promoter, a ubiquitin promoter, a globin promoter, an actin globin promoter, a phosphoglycerate kinase (PGK) globin promoter, a CAG promoter, a U6 promoter, a 7SK promoter, and an H1 promoter.

92. The kit of claim 91, wherein the promoter is selected from the group consisting of: the U6 promoter, the H1 promoter, and the 7SK promoter.

93. The kit of claim 75, wherein the kit comprises the sequence-targeting nuclease.

94. The kit of claim 75, wherein the kit comprises the second nucleic acid molecule. 95 The kit of claim 75, wherein the sequence-targeting nuclease is a polypeptide packaged into a liposome or LNP.

96. The kit of any one of claims 75-95, wherein the sequence-targeting nuclease is a Cas nuclease.

97. The kit of claim 96, wherein the Cas nuclease is about 400 amino acids to about 2000 amino acids in size.

98. The kit of claim 97, wherein the Cas nuclease is about 550 amino acids to about 1120 amino acids in size.

99. The kit of claim 96 or 97, wherein the Cas nuclease is selected from the group consisting of: a Cas9 nuclease, a Cas12a nuclease, and a Cas12f nuclease.

100. The kit of claim 99, wherein the Cas9 nuclease is from Sa or Sp.

101. The kit of claim 99, wherein the Cas12a or Cas12f nuclease is from Lb.

102. The kit of any one of claims 75-101, wherein the components are formulated for intravenous administration to the subject.

103. The kit of claim 102, wherein the intravenous administration is intravenous infusion to the subject.

104. The kit of any one of claims 75-103, wherein the blood disorder is selected from the group consisting of: a hemoglobinopathy, a primary immunodeficiency, a viral infection in the blood, a cytopenia, and a storage or metabolic disorder.

105. The kit of claim 104, wherein:
(a) the hemoglobinopathy is SCD or β-thalassemia;
(b) the primary immunodeficiency is X-SCID, ADA-SCID, WAS, or CGD;
(c) the viral infection is an HIV, HHV, or CMV infection in the blood;
(d) the cytopenia is FA or SDS; or
(e) the storage or metabolic disorder is Gaucher disease or X-ALD.
